(19) United States
(12) Patent Application Publication
(10) Pub. No.: US 2011/0152661 A1
(43) Pub. Date: Jun. 23, 2011

(54) Method and apparatus for identifying the viability of ischemic myocardium of a patient

(75) Inventors: Marc D. Feldman, San Antonio, TX (US); Anil Tharian George Kottam, London (CA); John Pearce, Austin, TX (US); Jonathan Valvano, Austin, TX (US); John Porterfield, Austin, TX (US)

(73) Assignee: Board of Regents, The University of Texas System

(21) Appl. No.: 12/924,195

(22) Filed: Sep. 22, 2010

Related U.S. Application Data
(63) Continuation of application No. PCT/US09/01808, filed on Mar. 23, 2009.
Application No. PCT/US09/01808 is a continuation-in-part of application No. 12/086,040, filed on Jun. 4, 2008.
(60) Provisional application No. 61/070,931, filed on Mar. 26, 2008.
(60) Provisional application No. 60/753,105, filed on Dec. 22, 2005.

(51) Int. Cl. A61B 5/0408; A61B 5/0402

(52) U.S. Cl. 600/393; 600/509

(57) ABSTRACT

A method for identifying the viability of ischemic myocardium of a patient. The method includes the steps of measuring in real-time the ischemic myocardium of a beating or non-beating heart. There is the step of determining in real-time whether the ischemic myocardium of the beating or non-beating heart is stunned or is nonviable. An apparatus for identifying the viability of ischemic myocardium of a patient's heart. The apparatus includes an electrode array having at least four electrodes for electrical communication with the heart which produces an array signal. The apparatus includes a processor portion in communication with the array which receives the array signal and determines in real-time whether the ischemic myocardium of the heart is stunned or is nonviable. An apparatus for analyzing living tissue. The apparatus includes an electrode array having at least four electrodes for electrical communication with the tissue which produces an array signal. The apparatus includes an admittance magnitude and phase detection circuit in communication with the array which receives the array signal from the array and produces a detection circuit signal corresponding to phase angle and magnitude of the array and signal. The apparatus includes a processor in communication with the detection circuit which receives the detection circuit signal and determines permittivity and conductivity of the tissue in real-time. A method for analyzing living tissue.

### CROSS-REFERENCE TO RELATED APPLICATIONS

This is a 371 of international application PCT/US2009/01808 filed Mar. 23, 2009, which is an international application of U.S. provisional application Ser. No. 61/070,931 filed Mar. 26, 2008. This is also a continuation-in-part of U.S. patent application Ser. No. 12/086,040 filed Jun. 4, 2008, which is a 371 of international application PCT/US2006/047649 filed Dec. 14, 2006, which claims priority from U.S. provisional application Ser. No. 60/753,105 filed Dec. 22, 2005, all of which are incorporated by reference herein.

### STATEMENT REGARDING FEDERALLY SPONSORED RESEARCH OR DEVELOPMENT

This invention was made with Government support under Grant No. HL079926 awarded by the National Institutes of Health. The Government has certain rights in the invention.

### FIELD OF THE INVENTION

The present invention is related to identifying the viability of ischemic myocardium of a patient by measuring in real-time the ischemic myocardium of a beating or non-beating heart to determine in real-time whether the ischemic myocardium of the beating or non-beating heart is stunned or is nonviable. (As used herein, references to the “present invention” or “invention” relate to exemplary embodiments and not necessarily to every embodiment encompassed by the appended claims.) More specifically, the present invention is related to identifying the viability of ischemic myocardium of a patient by measuring in real-time the ischemic myocardium of a beating or non-beating heart to determine in real-time whether the ischemic myocardium of the beating or non-beating heart is stunned or is nonviable based on permittivity and conductivity of the tissue.

### BACKGROUND OF THE INVENTION

Ischemic heart disease is the single leading cause of death in the United States and in the developed world. Overall, there are an estimated 71.3 million people in the United States living with cardiovascular diseases (CVD), which causes over 910,000 deaths annually compared to over 554,000 cancer deaths. In 2003, it was reported that 13.2 million Americans suffered from coronary heart disease (CHD) [58] and CHD was the cause of 1 in every 5 deaths in the country. The estimated direct and indirect cost of CHD was US $142 billion in 2006.

Ischemia is a restriction in blood supply caused by constriction or blockage of the blood vessels, resulting in tissue damage or dysfunction. Insufficient blood supply causes the tissue to become hypoxic or anoxic (if no oxygen is supplied at all). This can cause tissue necrosis, usually within 10-12 hours. Ischemia of the heart muscle results in a condition called angina pectoris which could result in a myocardial infarction. This can be due to atherosclerosis, thromboembolism, tachycardia, hypotension, or other causes.

Ischemia may also be inadvertently induced during certain surgical procedures or during the storage and transport of donor hearts prior to transplantation. This has an adverse effect on the cardiac cells, which determines the further viability of the heart. Hence any parameter that can monitor the onset and progression of myocardial ischemia, such as electrical admittance, could find widespread use in medicine as a real time ischemia detector.

Myocardial ischemia is known to change electrical properties of the myocardium [6, 41, 47, 48]. Both the conductive and capacitive behavior of myocardium change in response to an ischemic event. This may enable the real time monitoring of the onset and progression of ischemia by measuring the electrical conductivity and permittivity of the cardiac muscle.

Patients undergoing vascular surgery have a high risk of suffering major post operative cardiac events. There is an increased correlation of adverse cardiac events with the presence of perioperative myocardial ischemia [25, 44]. High risk patients display an increased frequency of tachycardia and asymptomatic ischemia in the first 48 hrs post operatively, with ischemia almost uniformly preceding adverse cardiac events [35, 43, 44]. All this has indicated the need for reliable ischemia monitoring. Urban et al. [59] have shown that clinically available hemodynamic indicators cannot identify intraoperative myocardial ischemia.

All measures of myocardial ischemia have their limitations. The commonly used evaluation techniques involve a 12-lead electrocardiogram (ECG) and cardiac markers such as troponins. A characteristic feature of ischemia is an elevation in the ST segment of the ECG. However, variations in ECG-ST segments produced by left ventricular hypertrophy, digitalis effect, and electrolyte abnormalities can mimic the ST segment changes of ischemia [59]. Cinca et al. [6] have recorded ST level elevation in electrodes overlying contiguous necrotic tissue, despite there being no ischemia of viable tissue in that region. Various bio-markers that are commonly used have their own limitations as well. Lactate dehydrogenase (LDH) is not specific to cardiac tissue whereas cardiac troponins are highly specific, but often very insensitive, requiring further testing to achieve an accurate diagnosis. All this further highlights the need for innovative methods of real time ischemia detection and monitoring.

Electrical impedance measurements have been used for more than 80 years to determine passive electrical properties of several tissues, including kidneys, brain, liver and spleen [33, 47, 56, 57]. The impedance is monitored for variations shortly after death or removal of the organ from the animal. The measurement of myocardial electrical impedance thus allows an overall estimation of the passive electrical properties of cardiac tissue.

It has been widely reported that the disruption of the plasma membrane is a characteristic feature of the final stage of hypoxic and ischemic necrotic cell death [23, 24, 37, 38, 47]. Two approaches are commonly used to determine the time course of cell death in myocardial ischemia or hypoxia [47]. The first one is based on the ability of some dyes to enter or exit the cell only if sarcolemmal integrity is lost. Methods based on this approach allow the monitoring of necrotic cell death in cell preparations but not in intact myocardial tissues [15, 38]. The second approach detects the presence of large intracellular molecules (LDH and troponin) in the extracellular space [15, 41, 42, 46]. These molecules cannot leave the cell while the plasma membrane is intact. This approach cannot be used during severe ischemia when the extracellular space in inaccessible.

Considerable research has been performed on the effects of ischemia on the electrical properties of myocardial tissue. The vast majority of literature focuses on changes of myocardial resistance or resistivity, rather than separating out the real and imaginary components of the electrical properties. Jain et al. [27] have measured a 400% increase in baseline resistance at 1 kHz during 50 min of ischemia in isolated rat hearts. del Rio et al. [8] showed that the time to plateau onset in dogs was longer than in pigs and humans, but the resulting ischemic plateau values in dogs, pigs and humans were not statistically different. Ivorra et al. [26] have made impedance and phase measurements on rat kidneys at 1 kHz. They have observed an impedance increase of 65% from the baseline value during 45 min of ischemia and a phase angle shift from −3.5° to −6.5° during the same period. Rodriguez-Sinovas et al. [46] have shown an increase in tissue resistivity from 150 Ω-cm to 320 Ω-cm during 45 min of ischemia in pig hearts. The resistivity returned to baseline within 15 min of reperfusion. The phase angle also shifted from −2° to −17° during the same period and returned to baseline almost immediately upon reperfusion. They made these measurements at a frequency of 7 kHz in both pig and isolated rat hearts. In rats, they report that ischemia induced a marked increase in myocardial resistivity (from 86.46±5.04 to 183.40±12.87 Ω-cm at 45 min of ischemia) and a marked negative shift in phase angle (from −0.36±0.26 to −4.62±0.43 degrees). Cinca et al. [6] have shown that resistivity of one month old myocardial infarction at 1 kHz was significantly lower than normal myocardium (110±30 vs. 235±60 Ω-cm) in pig hearts. They also show that there is a marked increase in tissue resistivity in the acute ischemic region after 60 min of proximal occlusion of the LAD and virtually no change in resistivity in the center of the necrotic scar.

The cell volume of virtually every human cell is regulated by a Na⁺/K⁺-ATPase, commonly known as the sodium potassium pump. In order to maintain the differential cell potential, cells must keep a low concentration of sodium ions and high levels of potassium ions within the cell. In the extracellular space, there are high concentrations of sodium and low concentrations of potassium, so diffusion occurs through ion channels in the plasma membrane. In order to keep the appropriate concentrations, the sodium-potassium pump pumps sodium out and potassium in through active transport. Hypoxia arising as a result of ischemia decreases the oxygen and hence the metabolic energy available to the cells, causing a failure of this active transport mechanism. As a result, the Na⁺ equilibrium is no longer regulated and an osmotic pressure imbalance develops between the intracellular and extracellular regions. The extracellular water penetrates into the cell with a subsequent cell swelling [13] and shrinkage of the extracellular region. This is manifested as a decrease in the electrical conductivity of the tissue at low frequencies [26].

Tissue necrosis caused by hypoxia is characterized by the breakdown of the structure and function of the plasma membranes of the hypoxic cells. The breakdown of the plasma membrane permeability barrier is preceded by a short metastable state that includes bleb formation, changes in lipid fluidity, and alterations in phospholipid composition [38, 47]. During this metastable state, blebs increase rapidly in size, trapped anionic fluorophores leak from the cytosol, onset of the mitochondrial permeability transition occurs, and lysosomes disintegrate [20, 23, 37, 38, 63]. The bleb finally ruptures and there is a rapid equilibrium of intracellular and extracellular contents [38]. Various high molecular weight intracellular proteins which were previously unable to leave the cell can now escape into the extracellular space. Such proteins include creatine phosphokinase (CPK), lactate dehydrogenase (LDH) and troponin which can all be used as biomarkers for cell necrosis.

Myocardial hibernation and stunning are two conditions caused by ischemia of cardiac tissue. Hibernation is defined as the reversible mechanical dysfunction of the heart during severe chronic ischemia [34]. Stunning, on the other hand, is the mechanical dysfunction that persists temporarily after reperfusion despite the absence of irreversible damage and despite restoration of normal or near normal coronary flow [2, 3]. The underlying biochemical mechanisms for stunning are still largely unknown although a number of hypotheses were proposed in the 1980s, most of which have been subsequently abandoned [3]. Presently, the two viable theories regarding the pathogenesis of myocardial stunning are the oxyradical hypothesis (generation of oxygen-derived free radicals) and the calcium hypothesis (Ca²⁺ overload and decreased responsiveness of myofilaments to calcium) [3].

The degree of myocardial dysfunction is one of the most potent predictors of future cardiovascular risk. Hence, it follows that the identification of stunned and/or hibernating myocardium can provide important information with regard to prognosis and clinical decision-making.

The major determinants of myocardial impedance are the extracellular and intracellular resistance, the gap junction conductance, and the cell membrane capacitance [6, 9, 19, 60]. Healthy myocardial tissue exhibits a marked frequency dependence in its impedance properties due to the high electrical permittivity of the cardiac muscle. Gabriel et al. [18] have suggested that the high permittivity most likely originates in the myocardial cellular trans-membrane charge distribution.

Myocardial electrical admittance (Y) is defined as the sinusoidal current (I) applied through the tissue divided by the voltage (V) measured across it. Due to the capacitive properties of the cell membrane, the myocardial tissue is not purely resistive. This causes a time delay between the voltage and current waves that can be determined from the phase angle of tissue admittance. The admittance (Y) is a complex number defined by

Y=G+jB (1)

B=2πfCₘ (2)

where G is the conductance (real part), B is the susceptance (imaginary part) and Cₘ is the muscle capacitance. Therefore, myocardial admittance can be precisely defined by two components: tissue admittance magnitude (|Y|) and phase angle (θ=tan⁻¹ B/G). If the myocardium is assumed to be a homogenous medium, an equivalent circuit model of the myocardium will comprise of the extracellular conductance in parallel with the cell membrane capacitance. The equivalent circuit is shown in FIG. 1. Gₘ is the extracellular conductance and Cₘ is the cell membrane capacitance.

A more accurate approach is to model the myocardium as an inhomogeneous medium. The tissue can be considered as a combination of three main elements, namely the intracellular region, the extracellular region and the cell membranes. The intracellular and the extracellular conducting media are arranged in parallel and cell membranes act as an admittance (conductor and capacitor) connecting them. In this case, the equivalent circuit model will comprise of the extracellular conductance in parallel with a series combination of the intracellular conductance and the cell membrane capacitance. Rodriguez-Sinovas et al. [47] have suggested that membrane rupture and colloid osmotic edema should reduce the total tissue resistance, considering the fact that both membrane rupture and colloid osmotic swelling have been suggested to occur almost simultaneously [38]. The equivalent circuit model for an inhomogenous medium is shown in FIG. 2. Gᵢ is the intracellular conductance.

From the electromagnetic point of view, Gₘ and Cₘ are calculated from:

$$\begin{matrix}{G}_{m} = \frac{I}{V} = \frac{\int \int J \cdot d A}{- {\int }_{L} E \cdot d l} = \frac{\int \int {σ}_{m} E \cdot d A}{- {\int }_{L}^{ } E \cdot d l} & ( 3 ) \\ {C}_{m} = \frac{Q}{V} = \frac{\int \int {ɛ}_{m} E \cdot d A}{- {\int }_{L} E \cdot d l} & ( 4 )\end{matrix}$$

where I is the current (A), Q is the charge (C), A is the surface enclosing the source electrode (m²) and L is the path length for potential calculation, σₘ is the myocardial conductivity (S/m) and ∈ₘ is the myocardial permittivity (F/m). It is assumed that both σₘ and ∈ₘ are constants in the myocardium. Then, combining equation (3) with (4) yields the conductance capacitance analogy.

$$\frac{{C}_{m}}{{G}_{m}} = \frac{{ɛ}_{m}}{{σ}_{m}} \qquad ( 5 )$$

Accordingly the myocardial conductance can be calculated from its capacitance in a fairly straightforward manner.

Coronary occlusion causes a rise in the myocardial resistivity and this has been reported in various species [4, 5, 6, 7, 10, 28, 40]. This rise in resistivity is due to the increase in extracellular and intracellular resistance that leads to cell-to-cell electrical uncoupling [28]. Ischemia causes an accumulation of intracellular Ca²⁺ [6, 7], reduction of ATP content [6, 55] and accumulation of amphipathic lipid metabolites [6, 62] in the ischemic myocardium. All this causes an increase in gap junction resistance resulting in cellular uncoupling [6].

### BRIEF SUMMARY OF THE INVENTION

The present invention pertains to a method for identifying the viability of ischemic myocardium of a patient. The method comprises the steps of measuring in real-time the ischemic myocardium of a beating or non-beating heart. There is the step of determining in real-time whether the ischemic myocardium of the beating or non-beating heart is stunned or is nonviable.

The present invention pertains to an apparatus for identifying the viability of ischemic myocardium of a patient's heart. The apparatus comprises an electrode array having at least four electrodes for electrical communication with the heart which produces an array signal. The apparatus comprises a processor portion in communication with the array which receives the array signal and determines in real-time whether the ischemic myocardium of the heart is stunned or is nonviable.

The present invention pertains to an apparatus for analyzing living tissue. The apparatus comprises an electrode array having at least four electrodes for electrical communication with the tissue which produces an array signal. The apparatus comprises an admittance magnitude and phase detection circuit in communication with the array which receives the array signal from the array and produces a detection circuit signal corresponding to phase angle and magnitude of the array and signal. The apparatus comprises a processor in communication with the detection circuit which receives the detection circuit signal and determines permittivity and conductivity of the tissue in real-time.

The present invention pertains to a method for analyzing living tissue. The method comprises the steps of producing an array signal from an electrode array having at least four electrodes in electrical communication with the tissue. There is the step of receiving the array signal from the array at an admittance magnitude and phase angle detection circuit in communication with the array. There is the step of producing a detection circuit signal corresponding to phase angle and magnitude of the array signal with the phase angle and magnitude detection circuit. There is the step of determining permittivity and conductivity of the tissue in real-time with a processor in communication with the detection circuit which receives the detection circuit signal.

### BRIEF DESCRIPTION OF THE DRAWINGS

FIG. 1 shows an equivalent circuit model of the myocardium (homogenous).

FIG. 2 shows an equivalent circuit model of the myocardium (inhomogenous).

FIG. 3 is a block diagram of the admittance magnitude circuit.

FIG. 4 shows an admittance magnitude calibration curve.

FIG. 5 is a block diagram of the analog phase system.

FIG. 6 is a circuit diagram of the first stage of the analog phase system.

FIG. 7 shows example waveforms for zero phase shift.

FIG. 8 shows example waveforms for 30 degree phase shift.

FIG. 9 is a circuit diagram of the second stage of the analog phase system.

FIG. 10 is a circuit diagram of the third stage of the analog phase system.

FIG. 11 shows PDF with a constant input of 10 degrees.

FIG. 12 shows PDF for several closely spaced phase inputs.

FIG. 13 shows actual phase vs. measured phase at 2 kHz.

FIG. 14 shows actual phase vs. measured phase at 30 kHz.

FIG. 15 shows actual phase vs. error at 2 kHz and 30 kHz.

FIG. 16 shows an instrument used to measure complex admittance.

FIG. 17 shows a front panel of the RamsES unit.

FIG. 18 shows a back panel of the RamsES unit.

FIG. 19 shows a surface probe used for admittance measurements.

FIG. 20 shows an experimental setup to measure field penetration depth of surface catheters.

FIG. 21 shows COMSOL simulation results for electric field distribution for tetrapolar electrode configuration.

FIG. 22 shows an epicardial surface probe used for admittance measurements.

FIG. 23 shows an epicardial surface probe and connector.

FIG. 24 shows field penetration results for σ=10,500 μS/cm.

FIG. 25 shows field penetration results for σ=1,850 μS/cm.

FIG. 26 shows |Y| vs. σ for the surface probe.

FIG. 27 shows a constant flow, non recirculating Langendorff system.

FIG. 28 shows measured LVEDP during 140 min of perfusion.

FIG. 29 shows developed pressure during 140 min of perfusion.

FIG. 30 shows an average heart rate during 140 min of perfusion.

FIG. 31 shows a heart rate of three control hearts during 140 min of perfusion.

FIG. 32 shows relative permittivity of myocardium (at 30 kHz) in control hearts.

FIG. 33 shows electrical conductivity in control hearts.

FIG. 34 shows average change in LDH release for the control hearts.

FIG. 35 shows measured LVEDP during 90 min of occlusion.

FIG. 36 shows relative permittivity of myocardium during baseline and 90 min of occlusion.

FIG. 37 shows electrical conductivity of myocardium during baseline and 90 min of occlusion.

FIG. 38 shows average increase in LDH release from baseline after 90 min occlusion.

FIG. 39 shows measured LVEDP during 15 min of occlusion and 95 min of reperfusion.

FIG. 40 shows developed pressure during 15 min of occlusion and 95 min of reperfusion.

FIG. 41 shows heart rate during 15 min of occlusion and 95 min of reperfusion.

FIG. 42 shows developed LV pressure in stunned vs. control hearts.

FIG. 43 shows LVEDP in stunned vs. control hearts.

FIG. 44 shows relative permittivity of myocardium during baseline, 15 min of occlusion and 95 min of reperfusion.

FIG. 45 shows normalized permittivity of myocardium during baseline, 15 min of occlusion and 95 min of reperfusion.

FIG. 46 shows electrical conductivity of myocardium during baseline, 15 min of occlusion and 95 min of reperfusion.

FIG. 47 shows average change in LDH release from baseline after 15 min occlusion.

FIG. 48 shows a comparison between myocardial permittivity and diastolic pressure during baseline and 90 min of occlusion.

FIG. 49 shows a comparison between myocardial conductivity and diastolic pressure during baseline and 90 min of occlusion.

FIG. 50 shows normalized permittivity of myocardium during baseline, 15 min of occlusion and 95 min of reperfusion (n=4).

FIG. 51 shows a comparison between normalized mean permittivity and LVEDP during baseline, 15 min of occlusion and 95 min of reperfusion (n=4).

FIG. 52 shows myocardial conductivity in stunned vs. control hearts (n=4).

FIG. 53 shows normalized relative permittivity—ischemia vs. stunning.

FIG. 54 shows normalized conductivity—ischemia vs. stunning.

FIG. 55 shows trends in myocardial permittivity and conductivity in normal, transient ischemic, stunned and recovered myocardium.

FIG. 56 shows trends in myocardial permittivity and conductivity during baseline, transient ischemia and diastolic dysfunction.

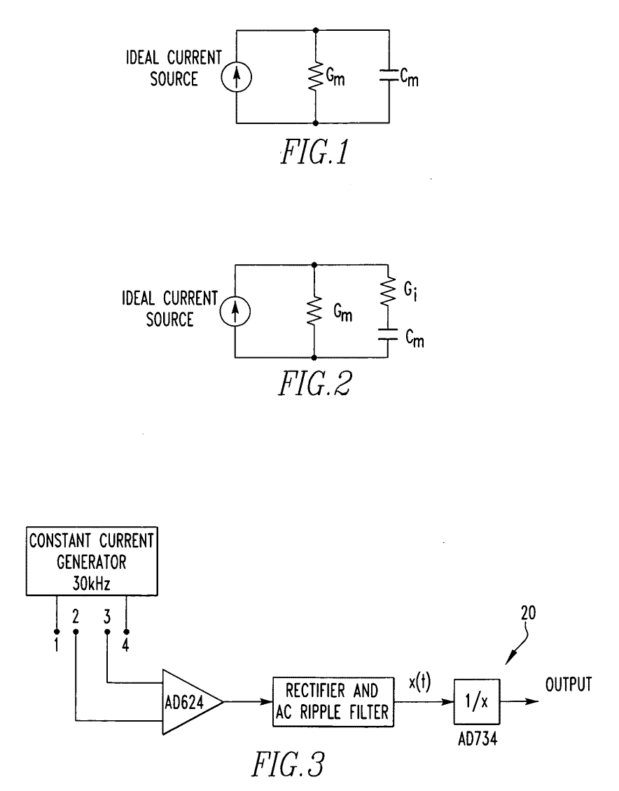

### DETAILED DESCRIPTION OF THE INVENTION

Referring now to the drawings wherein like reference numerals refer to similar or identical parts throughout the several views, and more specifically to FIGS. 3, 5, 6, 9, 10 and 16 thereof, there is shown an apparatus 10 for identifying the viability of ischemic myocardium of a patient's heart. The apparatus 10 comprises an electrode array 12 having at least four electrodes for electrical communication with the heart which produces an array signal. The apparatus 10 comprises a processor portion 14 in communication with the array 12 which receives the array signal and determines in real-time whether the ischemic myocardium of the heart is stunned or is nonviable.

Preferably, the heart is nonviable if its relative permittivity is greater than about 19,000.

The present invention pertains to an apparatus 10 for analyzing living tissue. The apparatus 10 comprises an electrode array 12 having at least four electrodes for electrical communication with the tissue which produces an array signal. The apparatus 10 comprises an admittance magnitude and phase detection circuit 16 in communication with the array which receives the array signal from the array and produces a detection circuit signal corresponding to phase angle and magnitude of the array and signal. The apparatus 10 comprises a processor 18 in communication with the detection circuit which receives the detection circuit signal and determines permittivity and conductivity of the tissue in real-time.

Preferably, the admittance magnitude detection circuit 20 is an analog phase angle and magnitude detection circuit 20. The admittance magnitude and phase angle detection circuit and the processor 18 together preferably are portable.

The present invention pertains to a method for identifying the viability of ischemic myocardium of a patient. The method comprises the steps of measuring in real-time the ischemic myocardium of a beating or non-beating heart. There is the step of determining in real-time whether the ischemic myocardium of the beating or non-beating heart is stunned or is nonviable.

Preferably, the measuring step includes the step of measuring the ischemic myocardium's electrical admittivity of the beating or non-beating heart.

The present invention pertains to a method for analyzing living tissue. The method comprises the steps of producing an array signal from an electrode array 12 having at least four electrodes in electrical communication with the tissue. There is the step of receiving the array signal from the array at an admittance magnitude and phase angle detection circuit in communication with the array. There is the step of producing a detection circuit signal corresponding to phase angle and magnitude of the array signal with the phase angle and magnitude detection circuit 20. There is the step of determining permittivity and conductivity of the tissue in real-time with a processor 18 in communication with the detection circuit which receives the detection circuit signal.

Permittivity of myocardium will rise if the muscle is being damaged. For permittivity to rise, large holes are occurring in the sarcolemma (myoctye membrane) so that large proteins like LDH can escape. These large holes will allow the electrochemical gradient to be lost as well. If these holes persist too long, then the damage will be permanent (about 90 min). If the damage is short (about 15 min), the damage is transient and can be repaired. Thus, a permittivity system could be used to evaluate viability or potential pending damage for hearts being transported, and to determine if cardioplegia is adequate during bypass surgery, valve replacement and repair, pediatric surgery, and any open heart operation where the myocardium is temporarily arrested. Accordingly, the apparatus 10 can also determine:
adequacy of cardio-plegia during open heart surgery. If the heart is arrested properly, it will not become necrotic during the operation. This would apply during CABG, valve replacement, and others;
viability of the human heart during transport for heart transplant, to be certain the heart is not ischemic and being damaged before transplantation into a new patient.

To the best knowledge of the inventors, the apparatus 10 is the first self contained portable device that measures admittance of the myocardium in real time. The measured admittance is used to calculate muscle conductivity and relative permittivity. Stunned myocardium will have normal conductivity and increased relative permittivity (15% increase from baseline, approx.). The relative permittivity of human myocardium is about 15,000 and so stunned myocardium would have a relative permittivity of approximately 17,250. Non viable myocardium will exhibit decreased conductivity (approx. 50% decrease from baseline) and increased relative permittivity (approx. 30% increase from baseline). This corresponds to a relative permittivity of about 19,000, and preferably 19,500 or greater for non viable human myocardium.

In the operation of the invention, the admittance magnitude measurement instrument is based on the system developed by Feldman et al. [11] redesigned to work at a single frequency of 30 kHz and is illustrated in FIG. 3. The desired excitation frequency of 30 kHz was generated using a 4 MHz crystal and a digital counter chip (CD 4040, Texas Instruments Inc., Dallas Tex.) to divide the crystal frequency by a factor of 128. The counter output was converted into a sinusoidal current signal that was applied to the two outer electrodes, #1 and #4 in FIG. 3. The instantaneous voltage signal between the inner electrodes, #2 and #3 in FIG. 3, was amplified with an instrumentation amplifier (AD 624, Analog Devices, Norwood Mass.). The AD624 was chosen for its high speed (fₜ=25 MHz), high common mode rejection ratio (CMRR=85 dB) and low noise characteristics (4 nV/√Hz). The signal was then actively rectified and filtered to remove the AC ripple. The constant DC voltage that is finally obtained is applied to a multiplier chip (AD 734, Analog Devices) whose output voltage is inversely proportional to the amplitude of the input signal and then scaled to ±10V to represent the conductance signal over the range of expected values. The output resolution of the magnitude measurement is 1 mV/μS.

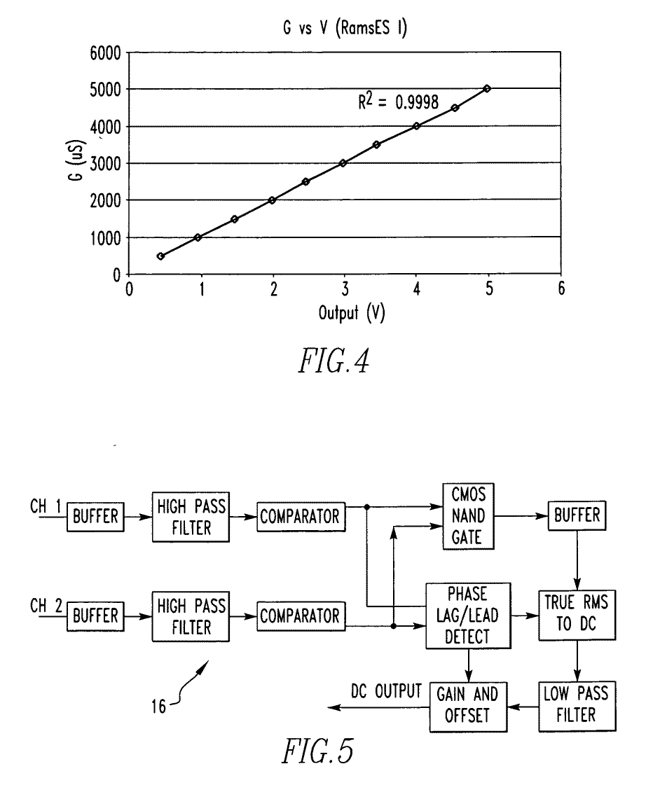

Calibration of the conductance measurement device was accomplished with 1% metal film resistors between 200Ω (5,000 μS) and 2.00 kΩ (500 μS). The calibration resistors were tested on an Agilent Inc. Model 4194A Impedance/Gain-Phase Analyzer to ensure that no inductive or capacitive behavior was observable in them over the frequency range of interest, 1 to 100 kHz. This curve is shown in FIG. 4. The miniaturized tetrapolar catheter cable has substantial inter-wire capacitance: there are six inter-electrode parallel capacitances among the four lead wires [30]. The net effect of these capacitances was compensated by calibration of the catheter by immersing in a relatively large volume of saline of known electrical conductivity. The conductivity of the saline solutions was measured with a Hanna Model HI 8033 conductivity meter (Hanna Instruments, Woonsocket R.I.) with the temperature compensation turned off. A calibration curve was generated (for every catheter that was used) at the measurement frequency of 30 kHz to cover the range of expected effective conductivities for muscle (2000 to 6000 μS/cm, or 0.2 to 0.6 S/m). These results are outlined in the following chapter.

The conductance measurement exhibits excellent linearity for input conductance varying from 500 μS to 5000 μS as seen in FIG. 4.

A phase detection system was designed and developed using discrete analog components that determines the phase angle between two input sinusoidal signals. The system generates an output DC voltage that is proportional to the phase difference between the input signals. The functional units in the phase detection network are illustrated in the block diagram of FIG. 5.

The high pass filters are first order filters with a cut off frequency of 33 Hz. They are used to eliminate any DC offset in the input signals. The high-speed comparators (LM 339, National Semiconductor Corp., Santa Clara Calif.) are used as zero crossing detectors and generate output square pulses varying from 0V to +5V with a duty cycle of 50%. These pulses are applied to the input of a NAND gate (CD 4011, Texas Instruments Inc., Dallas Tex.). The output of the NAND gate is a series of pulses whose duty cycle is proportional to the phase difference between the two input signals. A true RMS to DC converter chip (AD536, Analog Devices, Norwood Mass.) is used to obtain a DC voltage proportional to the duty cycle of the NAND gate output pulses. The low pass filter is a first order filter with a cut off frequency of 160 Hz and is used to remove any high frequency noise from the output of the true RMS chip. The gain and offset circuitry will enable the DC output of the circuit to vary from −9V to +9V for input phase angles varying from −90° to +90°. The phase lag/lead circuit is essentially comprised of a D-type flip-flop whose input and clock are connected to the two comparator outputs. The output of the flop will be either 0V or +5V depending on whether the sine wave in channel 1 leads (+5V output) or lags (0V output) the wave in channel 2. This flop output is used to set the polarity of the final DC output of the system.

The phase circuit has a sensitivity of 100 mV/degree. The measured phase angle is corrected to remove catheter and measurement system phase shift effects by making phase angle measurements using the same catheter in saline of known conductivity. Since the phase shift in saline has to be zero degrees, any measured phase shift is the effect of the catheter and the measurement system. For small phase angles (0 to 5°), there is a non-linearity in the DC voltage output of the system. This does not affect the measurements since the phase from the catheter and measurement system is between 30 and 35 degrees, ensuring that the system never has to differentiate between phase angles smaller than 5 degrees.

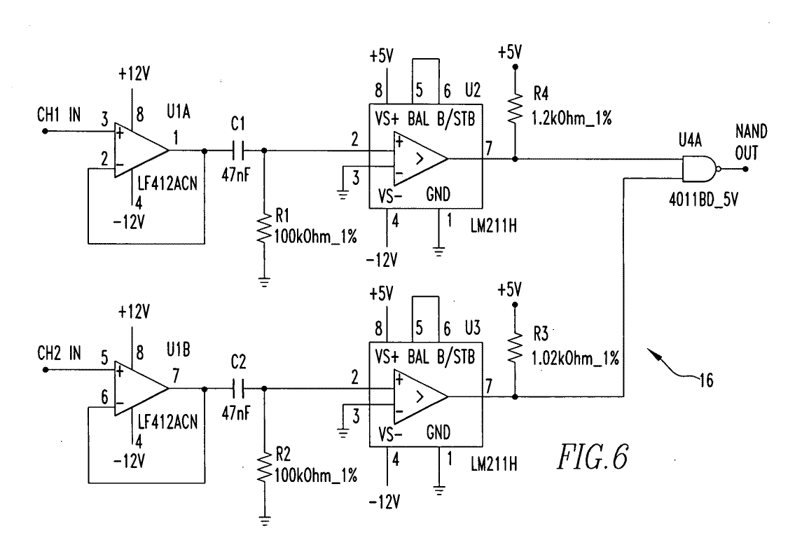

The first stage includes high pass filters, comparators and the CMOS NAND gate, shown in FIG. 6.

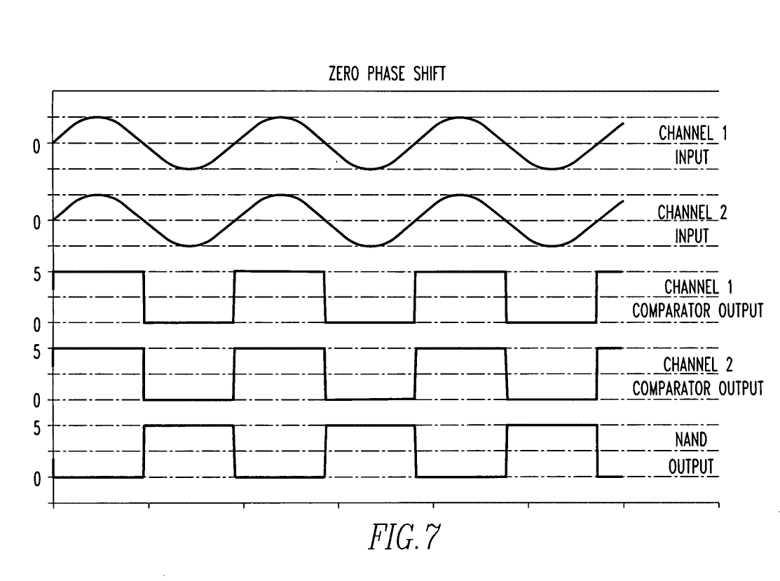

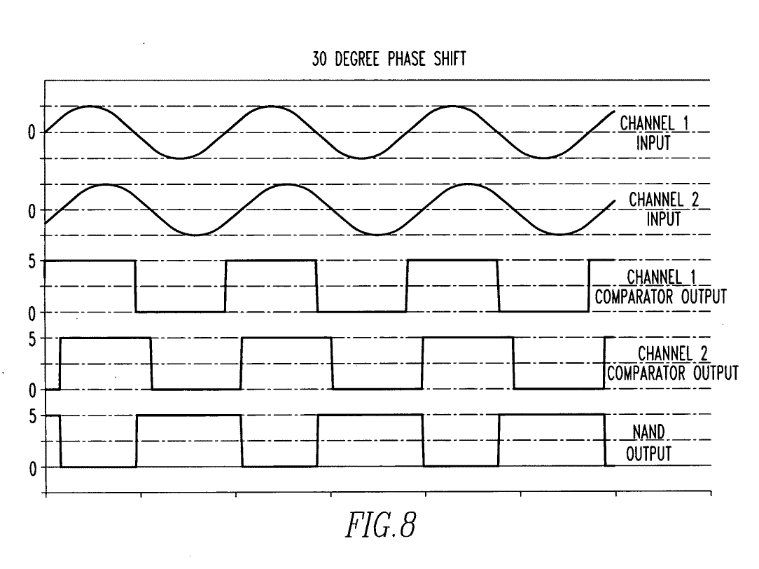

The high pass filters are passive first order filters with a cut off frequency of 33 Hz. The high speed comparators (LM211) convert the input sinusoidal signals into square waves which are applied to the two inputs of a CMOS NAND gate (CD4011). The NAND gate output is a 50% duty cycle square wave if the input signals to channels 1 and 2 are in phase. This duty cycle increases to 100% as the phase angle between the input channels increases to 180°. This is illustrated in FIGS. 7 and 8.

A more efficient implementation would have been to use an XOR gate instead of a NAND gate, since the square wave output duty cycle of the XOR gate would vary from 0% to 100% corresponding to input phase angles of 0° and 180°, respectively. However, when the XOR gate is used in combination with the true RMS to DC chip, the output is non linear for small values of phase angle when the duty cycle has to increase from zero to one percent. Thus the NAND gate was selected over the XOR gate in the final design.

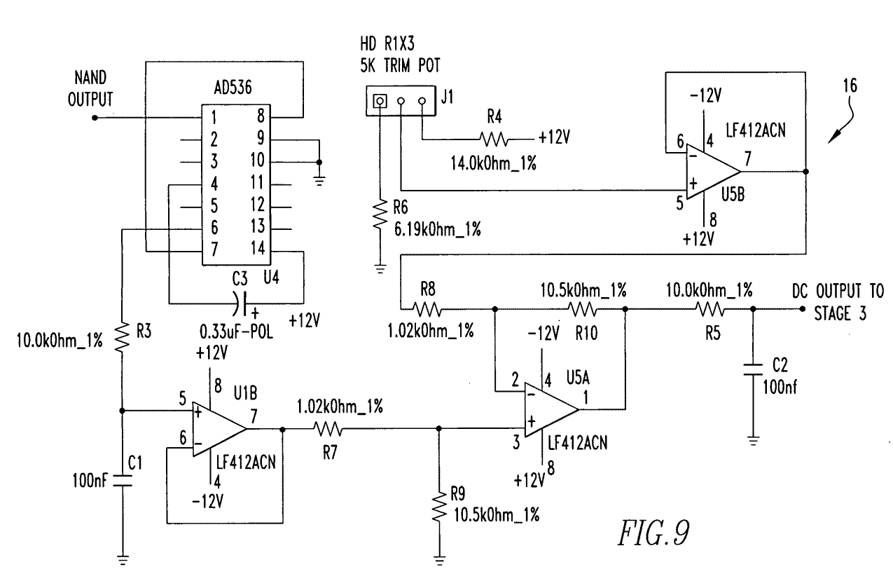

The second stage, shown in FIG. 9, includes the true RMS to DC conversion circuit, low pass filter and a gain and offset adjust circuit. The NAND output from the first stage is connected to the input of the true RMS to DC chip (AD536). The output of this circuit is a DC voltage level dependent on the duty cycle of the NAND output. The following low pass filter has a cut off frequency of 160 Hz to remove high frequency noise spikes from the DC output. The gain circuit consists of a summing amplifier that adds an offset voltage to the AD536 output so that a phase angle of 0° corresponds to 0V and a phase angle of 90° corresponds to +9V. The offset voltage is adjustable to calibrate out the phase effects of the instrumentation. The final output of this stage is a DC voltage ranging from 0 to +9V for phase angles varying from −90° to +90°. This stage cannot distinguish between positive and negative phase angles. This is accomplished in the third and final stage.

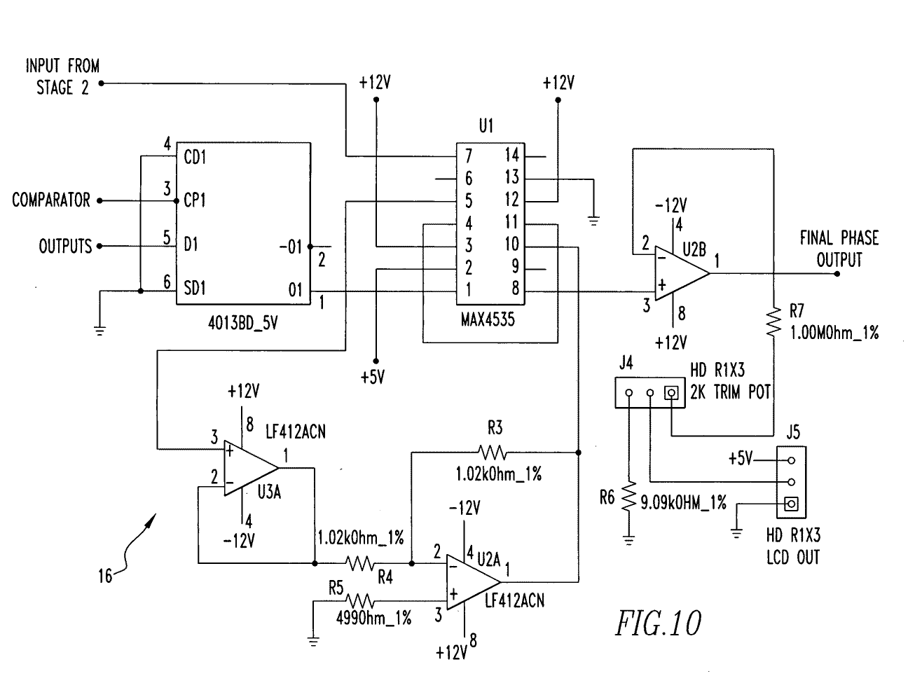

The final stage consists of the phase lag/lead detect circuit. This is accomplished using a D-type flip-flop whose input and clock are connected to the two comparator outputs. The output of the flop will be either 0V or +5V depending on whether the sine wave in channel 1 leads or lags the wave in channel 2. This flop output is used as one of the control signals of an analog switch (MAX4535) that routes the DC phase signal through either a buffer or a buffer and a unity gain inverting amplifier that inverts the polarity of the DC signal. Hence the final DC output of the system varies from −9V to +9V for phase angles varying from −90° to +90°. The final output is also scaled down by a factor of 100 and connected to an LCD panel meter to display the phase angle in degrees. The detailed circuit diagram for this stage is shown in FIG. 10.

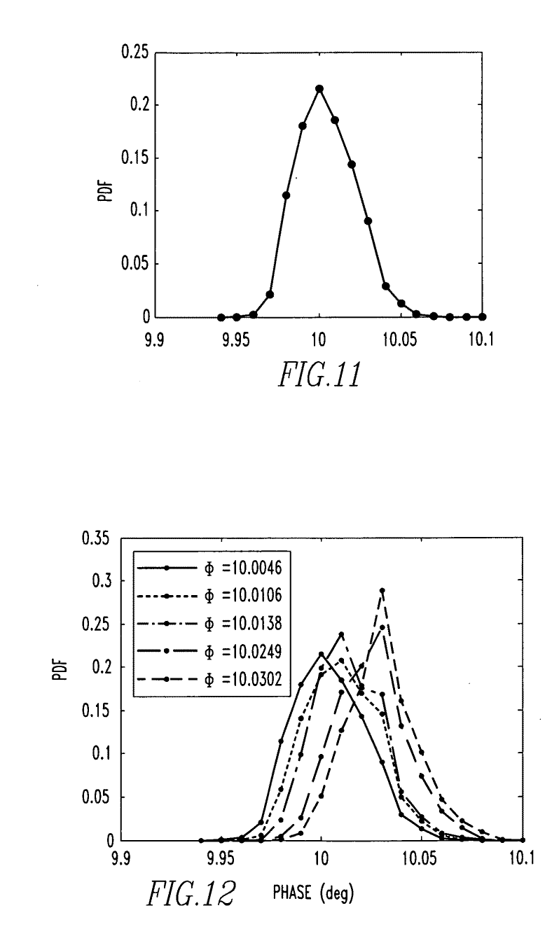

The noise analysis of the phase system was performed by taking a large number of measurements at a constant input phase difference of ten degrees. The probability density function (PDF) of approximately 3,500 output samples was acquired using an ADInstruments Powerlab data acquisition system and plotted as shown in FIG. 11.

The noise is extremely low, ±0.05 degrees. The PDF of the distribution is very close to a Gaussian distribution and the noise has a variance of 0.006 degrees.

A simple method to estimate resolution is to measure the peak-to-peak variations resulting with a fixed input, as shown in FIG. 11. Given this definition, the phase resolution is 0.1° because the typical variations are ±0.05°.

The system resolution was also determined by acquiring data for several closely spaced phase offsets. A Students-T test was performed for various numbers of samples. The PDF of several phase offsets is shown in FIG. 12 where each PDF was calculated using approximately 3,500 samples.

To use the t-statistic, one assumes the errors are normally distributed (with equal variances), and the errors are independent within each group and between the groups. Phase data is first collected for φ=10.0046°. Then, the phase is changed and phase data is collected again. The null hypothesis is that the means of two normally distributed populations are equal (meaning the instrument cannot resolve the change). For a sample size of 20, the t-statistic must be greater than 2.1 for a 95% confidence interval to reject the null hypothesis (meaning the instrument can resolve the change). Table 1 shows the calculated t-statistic for various numbers of samples. From the table, it is seen that the resolution for a sample size of 20 is about 0.03° (since the t-statistic between φ=10.0046° and φ=10.0304° is greater than 2.1). This value is slightly smaller than the system accuracy, which is at best 0.1°.

TABLE 1
t-statistic values for various numbers of samples, taken
between φ = 10.0046° and the other four.

| Sam- | t-statistic | | | |
|---|---|---|---|---|
| ples | φ = 10.0106° | φ = 10.0138° | φ = 10.0249° | φ = 10.0302° |
| 20 | 0.63 | 2.52 | 0.08 | 4.69 |
| 100 | 1.80 | 0.40 | 5.76 | 12.25 |
| 200 | 3.13 | 1.69 | 9.75 | 17.07 |

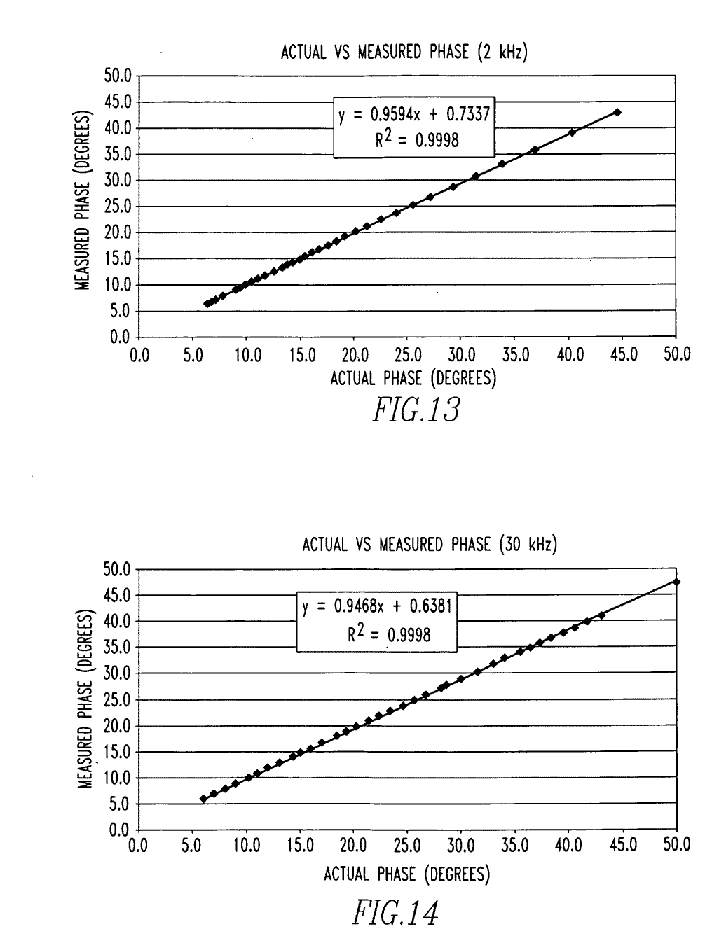

The phase accuracy of the system was determined by applying sinusoidal signals with a known phase difference at the input and measuring the system output. The input phase difference was calculated using an oscilloscope. The measurements were made at 2 kHz and 30 kHz and the results are shown in FIGS. 13 and 14.

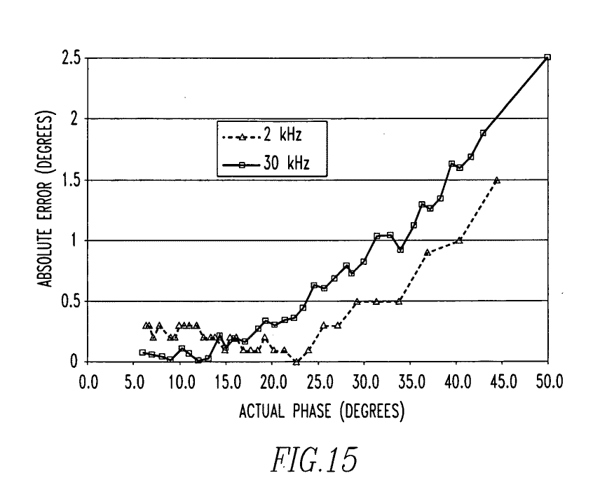

It is evident from these graphs that the instrument is linear over all regions analyzed for both 2 kHz and 30 kHz signals. In both cases the correlation coefficient, R², is 0.9998. Absolute error was then calculated as the absolute value of the difference between the phase measured by the instrument and the actual phase, as shown in FIG. 15.

It should be noted that this error is due to both error in the instrument and the error in the estimation of the actual input phase angle. Previous estimates of phase angle in the mouse myocardium using digital signal processing of the excitation and measured signals have shown phase angles between 5 and 30 degrees [29]. The instrumentation to measure admittance magnitude will induce a phase offset of about 10 degrees (at 30 kHz) from the band pass filters used in the circuit and the inter-wire capacitances in the surface probe.

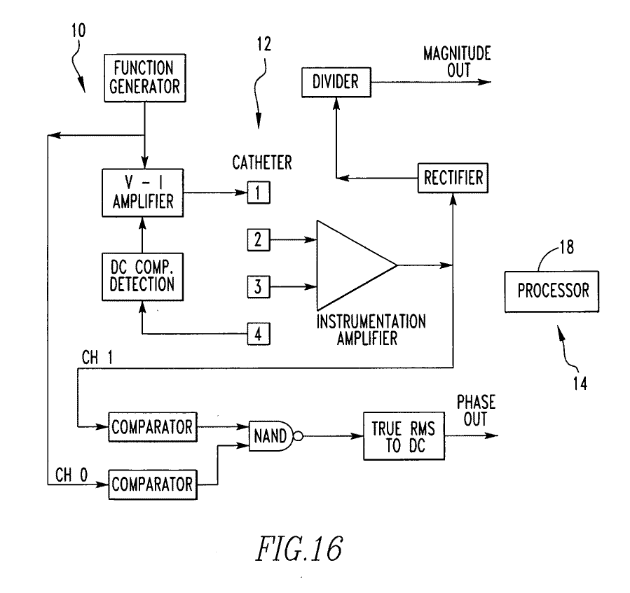

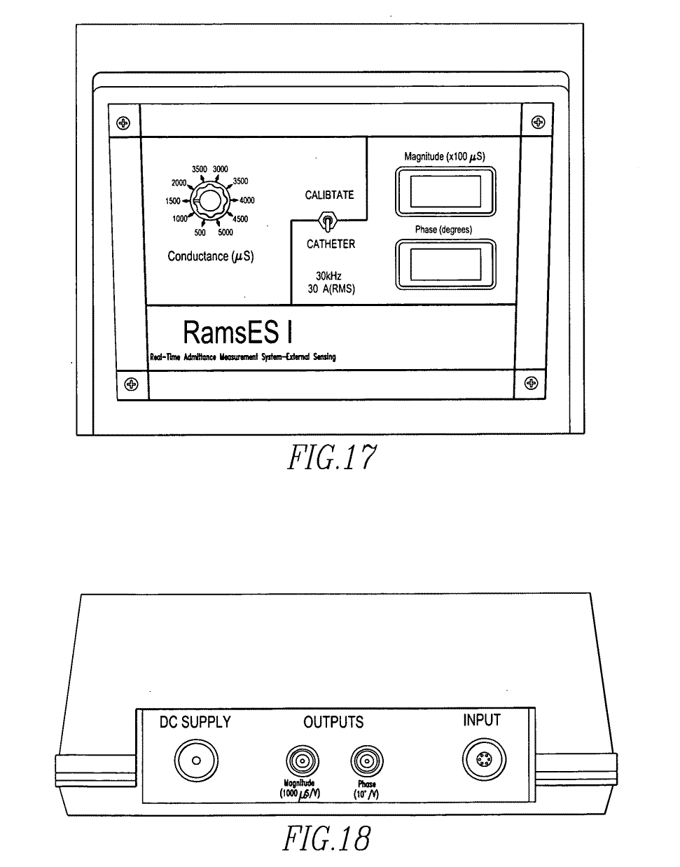

The admittance magnitude and phase measurement systems described earlier are combined into a single integrated system. The block diagram of the system is shown in FIG. 16.

The RamsES I system has two BNC analog output channels, one each for the magnitude and the phase angle. These two parameters are also displayed on two LCD panel meters located on the front panel of the instrument. The system is designed to work over an admittance magnitude range of 500 μS to 5000 μS and phase angle range of −90° to +90°, which covers the range of values measured in the myocardium as well as in the LV blood pool. A calibration switch is used to switch between the internal conductance calibrator and the external catheter. The catheter is plugged in via a six pin Redel™ connector (LEMO connectors). The system is powered using an external low-noise plug-in power supply manufactured by Stancor Electronic (Star-9007). The supply provides up to 1 A at 5V and 300 mA at ±12V. The RamsES I circuit draws approximately 100 mA from the ±12V supply and about 20 mA from the 5V supply, which is well within the limits of the power supply. FIGS. 17 and 18 show the front and back panels, respectively, of the system.

The use of tetrapolar electrode configuration for admittance measurements and its advantages over the bipolar electrode configuration have been described elsewhere [51, 54]. Briefly, the tetrapolar configuration is preferred since it compensates for the electrode-electrolyte interface impedances. In a linear tetrapolar electrode configuration, a constant current is injected through the outer electrode pair and the voltage difference between the inner electrode pair is measured. Since the current is kept constant, Ohms law dictates that any change in the measured voltage corresponds to a change in the admittance between the inner electrode pair. FIG. 19 shows a surface probe used for admittance measurements.

Myocardial tissue is highly anisotropic due to the complex structure of fiber orientation. There is a change in fiber orientation by over 120° between epicardium and endocardium. This anisotropy of the myocardial tissue makes it necessary to use a surface probe that has a depth of penetration approximately equal to the thickness of the myocardium to ensure that the admittance measurements are isotropic. The field penetration depth is directly proportional to the electrode spacing on the probe.

The experimental technique to determine the field penetration depth of tetrapolar surface admittance catheters is described by Kottam et al. [31]. Briefly, the experimental setup consists of a flat bottomed dish filled with saline with a movable plastic sheet at the bottom. The position of the movable sheet could be accurately adjusted by means of a micro manipulator with a resolution of 0.1 mm. The admittance catheter was placed on the saline surface and the plastic sheet was moved closer to the catheter. The admittance magnitude was monitored continuously and the depth of penetration was determined to be the spacing between the catheter and the plastic sheet that caused the admittance magnitude to drop to ninety percent of the magnitude when the plastic sheet is at the bottom of the dish (i.e. |Y|_{inf}). The experimental setup is shown in FIG. 20.

The measured depth of penetration was verified using finite element models developed using a commercially available software package (COMSOL). The simulation results were compared with the actual in vitro experimental results in saline of known conductivities. This also enabled the estimation of the probe constant Kₚ for the different catheter designs. The models indicate that the effective penetration depth increases as the spacing between the sensing electrodes (#2 and #3) is increased. If this spacing is kept constant, the effective penetration depth is affected by the spacing between the stimulation and the sense electrodes. This is because the field geometry is determined only by the spacing between the two outer stimulation electrodes (#1 and #4) and the penetration depth decreases as this spacing is decreased. The results are summarized in Table 2. The calculated penetration depths were also independent of the conductivity of the bulk medium in the models.

TABLE 2
Electric field penetration for various electrode spacing
(COMSOL results)

| Electrode Spacing (mm) | | | Calculated field |
|---|---|---|---|
| 1-2 | 2-3 | 3-4 | penetration depth (mm) |
| 0.75 | 1.6 | 0.75 | 1.73 |
| 0.75 | 2.2 | 0.75 | 1.93 |
| 0.75 | 2.0 | 0.75 | 1.87 |
| 0.5 | 2.0 | 0.5 | 1.41 |
| 0.5 | 1.8 | 0.5 | 1.33 |

The current density over the electrode surface is not uniform, but is denser around the edges of the electrode. Using a constant current boundary condition at the electrodes will force the current density to be uniform over the entire electrode surface in the model. To overcome this, the stimulation electrodes are specified as constant potential electrodes with an excitation voltage of ±1.5V at 31.25 kHz. The electric field distribution for a tetrapolar electrode configuration with constant voltages of ±1.5V applied between the outer electrodes is shown in FIG. 21. In this case, the bulk medium is assumed to be isotropic with a conductivity of 2000 μS/cm.

The admittance was measured between electrodes 2 and 3 for different distances of the bottom insulating layer from the tetrapolar electrode surface. The distance was decreased from 5 mm down to 0.5 mm and the distance at which the measured admittance dropped to 90% of the value at 5 mm was considered as the effective penetration depth.

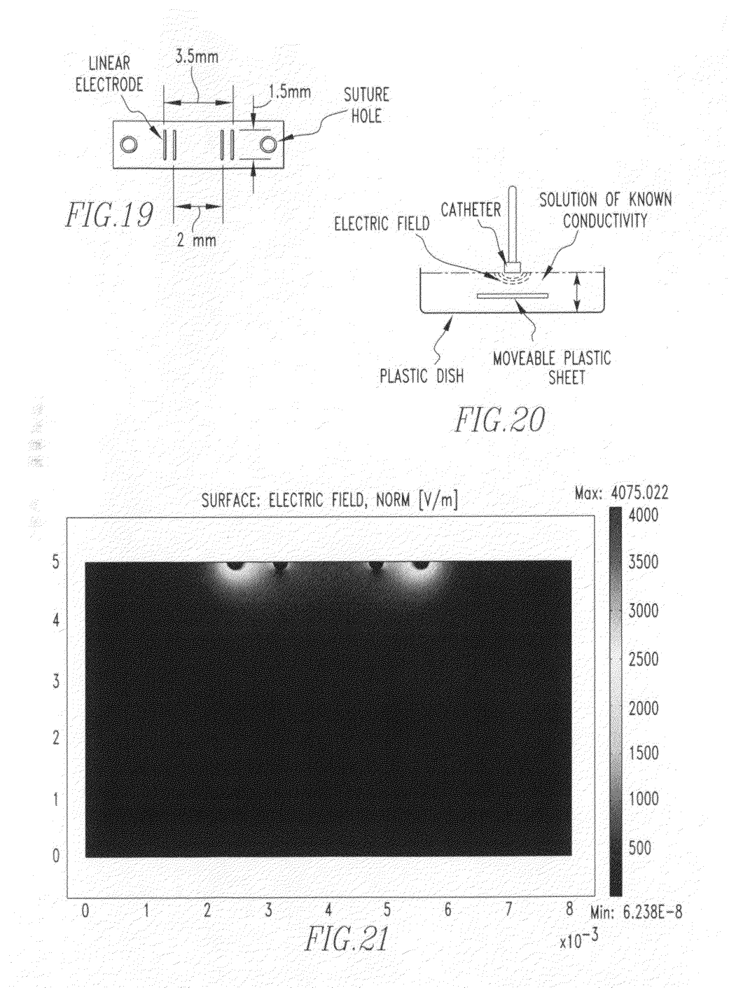

A tetrapolar admittance probe was designed and built that can be sutured onto the epicardial surface of the left ventricle of rat hearts. This probe is used to measure localized myocardial admittance during baseline, varying durations of ischemia and reperfusion. The surface probe is comprised of four linear electrodes mounted on a polystyrene strip. The electrodes are made from 0.4 mm diameter solid gold wire (Cooner Wire, part no. CW 5263-26). The electrode spacing is illustrated in FIG. 19. FIG. 22 shows the epicardial surface probe used for admittance measurements.

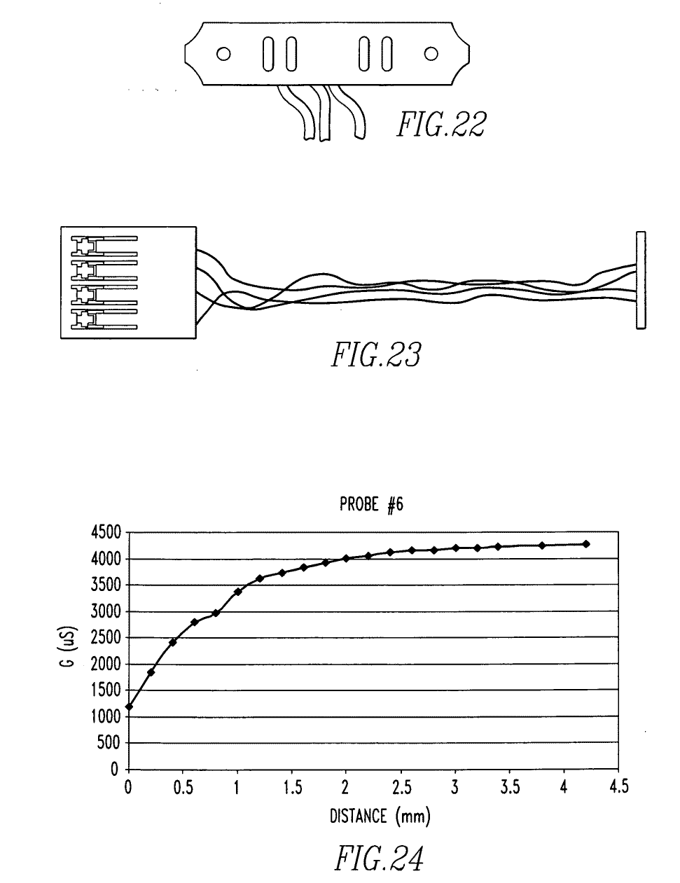

The electrodes were attached to the polystyrene strip using fast acting cyanoacrylate glue. The other ends of the gold wires were connected to a four pin Molex header connector. The entire catheter and connector assembly is shown in FIG. 23.

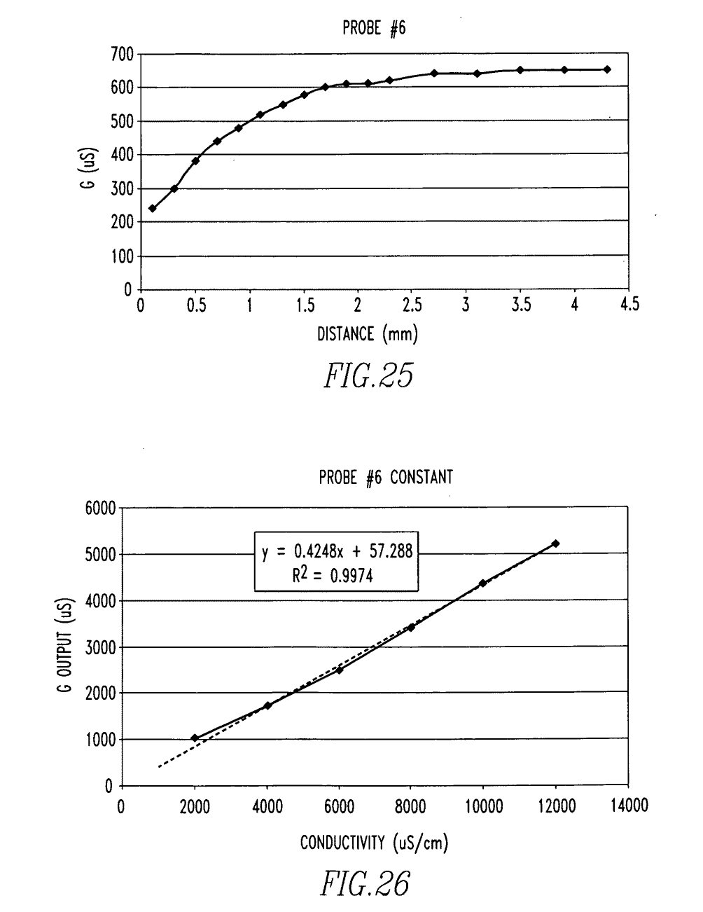

The electric field penetration depth of this probe was determined experimentally using the technique described above. Two different saline solutions with conductivities of 10,500 μS/cm and 1,850 μS/cm were used to ensure that the measurement results were independent of the saline conductivity. The measurement results for one of the probes are shown in FIGS. 24 and 25. The field penetration depth was calculated to be 1.6 mm in both cases. The COMSOL simulation results show a field penetration depth of 1.87 mm for the same electrode spacing. This is because the COMSOL models are in 2D and assume an infinite electrode length along the third dimension. In reality, the finite electrode length causes the field lines to constrict around the ends of the electrode. Thus the model tends to overestimate the field penetration depths when compared with the experimental results.

The left ventricular wall thickness of rats ranges from 1.6-2.0 mm for body weights ranging from 200 to 300 grams [39]. A total of three surface probes were built and characterized with differing electrode spacing. The measured and calculated field penetration depths for all three probes are shown in Table 3.

TABLE 3
Measured vs. theoretical field penetration results for three surface probes

| | |
|---|---|
| Electrode | |
| Spacing (mm) | Penetration Depth (mm) |

| Probe # | 1-2 | 2-3 | 3-4 | Experimental | Calculated |
|---|---|---|---|---|---|
| 6 | 0.75 | 2.0 | 0.75 | 1.60 | 1.87 |
| 7 | 0.50 | 1.8 | 0.50 | 1.15 | 1.33 |
| 8 | 0.75 | 1.6 | 0.75 | 1.47 | 1.73 |

From the above table, it is evident that probe #6 the optimal choice for the myocardial properties measurement (field penetration depth almost equal to LV wall thickness). The length of the probe must also enable all four electrodes to be in contact with the tissue without having to bend the probe to conform to the curvature of the heart.

The probe constant K is defined as the ratio between the actual conductivity (a) of the medium and the conductance (G) measured by the probe. Its value depends on the relative spacing of the electrodes on the probe which affect the field geometry.

σ=K×G (6)

In terms of the permittivity of the medium (∈ₘ) and the capacitance (C) measured by the probe,

∈ₘ=K×C (7)

From equation 3 described in chapter 1, the probe constant K can be related to the field form factor F as follows.

$$K = \frac{σ}{G} = \frac{{ɛ}_{m}}{C} = \frac{- {\int }_{L} E \cdot d l}{\int \int E \cdot d A} = \frac{1}{F} \qquad ( 8 )$$

The probe constant was determined by making conductance measurements on saline of known conductivities. These calibration solutions were prepared by mixing calculated amounts of sodium chloride in distilled water. Six different calibration solutions with conductivities of 2000, 4000, 6000, 8000, 10000 and 12000 μS/cm were prepared and the conductivities were verified using a commercially available conductivity meter (Hanna Instruments) with the temperature compensation turned off.

The known conductivities were plotted on the x axis and the measured admittance magnitudes were plotted on the y axis. This graph is shown in FIG. 26. The probe constant K is calculated by taking the reciprocal of the slope of the line. For the probe shown above (probe #6), value of K is 2.35 cm⁻¹.

The most prevalent experimental model to study myocardial ischemia and stunning is the Langendorff preparation of isolated perfused heart. The Langendorff model has been used in cardiovascular research for over 40 years [22] and provides a highly reproducible model that can be studied quickly and in large numbers at low cost. The effects of myocardial ischemia can be studied more easily under controlled conditions in isolated models.

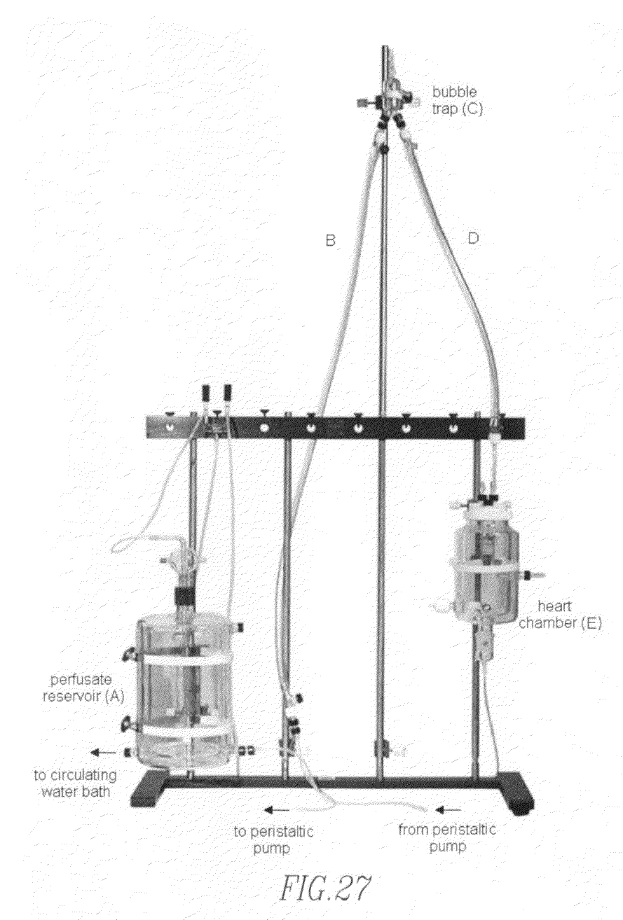

The isolated heart can be perfused in either constant flow or constant pressure mode. Constant flow perfusion adds an additional element of constancy to the experiment and simplifies the analysis of experimental data. The disadvantage here is that unlike in a constant pressure system, autoregulatory mechanisms are overridden and the system does not automatically alter the amount of perfusate delivered to the whole heart when there are changes in heart rate or load or when regional ischemia is imposed [22]. In spite of this disadvantage, it is experimentally preferable to control flow rate and heart rate since these variables can influence other indices of mechanical function. A constant flow, non recirculating model was used (Radnoti Glass Technologies Inc.) for the ischemia/stunning studies. The system is shown in FIG. 27.

The system uses a water jacketed 2-liter reservoir (A) to maintain and oxygenate the perfusate solution. A peristaltic pump draws perfusate from the reservoir and drives the solution up to a water-jacketed bubble trap (C) via a water jacketed flex tube assembly (B), where bubbles are removed from the flow, and then down to the aortic cannula into the heart chamber via a second water jacketed flex tube assembly (D). Effluent from the heart can be collected for evaluation. The constant flow peristaltic pump is adjustable with flow rates ranging from 1-10 ml/min. A pump-driven circulating water bath is used to circulate warm water through the water jackets on the tubes and glass reservoirs for excellent temperature control of the buffer. Myocardial stunning is induced in this model by 15 min of global no-flow ischemia.

The temperature of the myocardial surface must be monitored and regulated since conductance is a function of the temperature of the tissue. This was achieved by maintaining the perfusate at 36° C. and placing the isolated heart in a water jacketed temperature regulated heart chamber (E). The relation between temperature and conductivity is given by

σₜ=σ₀ exp [α(t−t₀)] (9)

where σₜ is the conductivity at temperature t, σ₀ is the conductivity at temperature t₀ and α is the temperature coefficient of conductivity (0.021° C.⁻¹ for NaCl).

The perfusion fluid that is most commonly used in Langendorff studies is the Krebs-Henseleit bicarbonate buffer. This fluid mimics the key ionic content of blood and plasma and has a pH of 7.4 at 37° C. The effluent from the heart can be collected at regular intervals and analyzed for elevated LDH levels, which is an indicator of ischemic cell death.

The various steps involved in setting up the constant flow, non recirculating Langendorff system are outlined below.
Circulate distilled water through perfusate channels for 15 min to flush out the system.
Insert Millar pressure transducer above the aortic cannula (pressure should be 50-100 mmHg).
Run the water bath at ˜42° C. and circulate the warm water through the water jacket tubes. Verify that the temperature of liquid in perfusate channels is 37° C.
Adjust perfusate flow rate to 10 ml/min.
Attach LV balloon to the end of the fluid filled LV catheter, taking care to remove any trapped air bubbles.
Prepare Krebs-Henseleit (KH) bicarbonate buffer by mixing the following amounts of salts in 1 litre of water. The calcium should be added at the very end after ensuring that the perfusion solution has a pH of 7.40.

| | |
|---|---|
| NaCl (118.5 mM) | 6.900 gms |
| NaHCO₃ (25 mM) | 2.100 gms |
| KCl (4.7 mM) | 0.350 gms |
| MgSO₄ (1.2 mM) | 0.296 gms |
| KH₂PO₄ (1.2 mM) | 0.163 gms |
| glucose (11.0 mM) | 1.982 gms |
| CaCl₂ (1.4 mM) | 0.155 gms |

If the pH is greater than 7.40, add a few drops of hydrochloric acid to reduce the pH.
Pour into the buffer into the perfusate container of the Langendorff system.
The buffer will be saturated with gas (95% O₂/5% CO₂) through a bubbler inserted into the perfusate container.

The isolated heart studies were done on adult male WKY rats weighing between 210 and 300 gms. The protocol that was used for the ischemia and the stunning studies is outlined below.
Weigh the rat after it is anesthetized using the standard rat cocktail.
Rapidly excise the heart via anterior thoracotomy and place in ice-cold (4° C.) beaker of Krebs-Henseleit (KH) bicarbonate buffer to arrest the heart.
Perform aortic cannulation rapidly (within 60 seconds) and perfuse the hearts with KH buffer in Langendorff mode at a flow rate of 10 ml/min.
Remove left atrial appendage to provide a clear field of view.
Insert a deflated balloon into the LV via the mitral valve. This balloon is attached to a short rigid catheter and a Millar pressure transducer.
Inflate the balloon with water from a micro syringe until an LVEDP of between 4 and 8 mmHg is obtained.
Position the tetrapolar surface probe on the epicardial surface, above the LV. Monitor admittance magnitude and phase angle continuously using the RamsES I unit.
Throughout the experiment the heart will be placed in a water-jacketed chamber (E) to maintain constant temperature during the ischemic period.
Monitor aortic pressure throughout the working heart model perfusion periods in the aortic outflow line with a Millar transducer.
Allow the isolated heart preparation to stabilize for about 10 min.
Collect baseline perfusate samples.
The isolated heart system is setup as described above.
Hang the heart and perfuse for 140 min.
Draw perfusate samples at three, five and ten minute intervals during perfusion.
Analyze the samples for markers of ischemia (LDH).
Weigh the heart, make slices along the short axis and store the slices in formalin for histologic analysis.
The isolated heart system is setup as described above.
Induce global no-flow ischemia by stopping aortic perfusion for 90 min.
Reperfuse for 20 min by reestablishing aortic perfusion.
Draw perfusate samples at five minute intervals.
Analyze the samples for markers of ischemia (LDH).
Weigh the heart, make slices along the short axis and store the slices in formalin for histologic analysis.
The isolated heart system is setup as described above.
Induce global no-flow ischemia by stopping aortic perfusion for 15 min.
Reperfuse for 90 min by re-establishing aortic perfusion.
Draw perfusate samples at three, five and ten minute intervals during reperfusion.
Analyze the samples for markers of ischemia (LDH).
Weigh the heart, make slices along the short axis and store the slices in formalin for histologic analysis.

Three adult male WKY rat hearts were perfused in constant flow Langendorff mode according to the protocol described in the earlier section. The measured admittance magnitude and phase angle was used to determine the relative permittivity and conductivity of the myocardial tissue over a perfusion duration of 140 min. LDH samples were also collected at regular intervals to determine the extent of myocardial cell damage, if any. These hearts served as controls and the results were compared with similar results from hearts subject to the stunning and ischemia protocols.

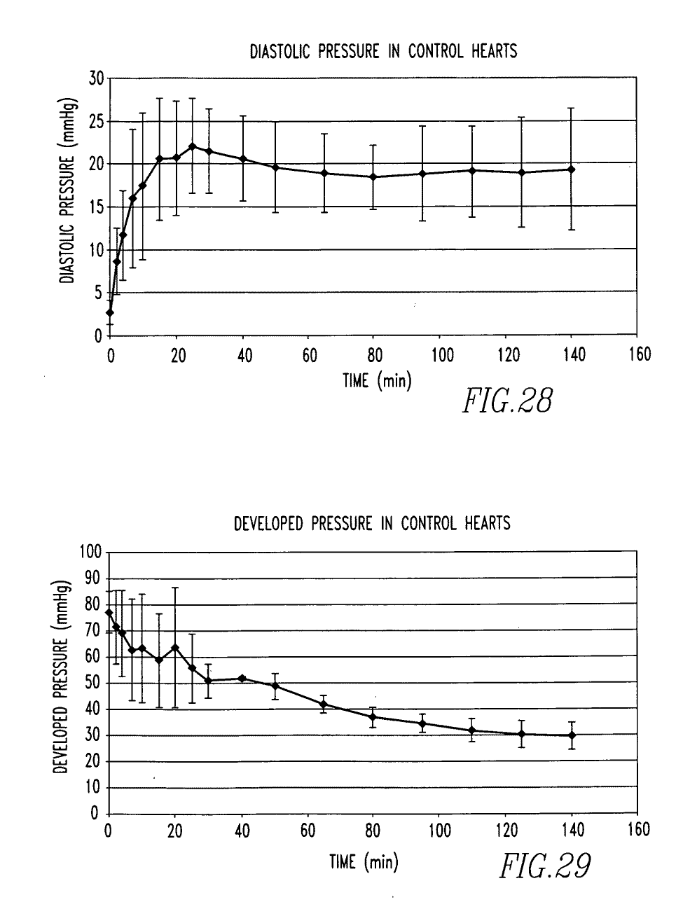

The LV pressure was monitored by inserting a fluid filled balloon into the LV. The pressure in the balloon was measured using a 1.4 Fr. Millar pressure sensor and was zeroed prior to insertion into the LV. After insertion, the balloon was inflated/deflated to obtain a left ventricular end diastolic pressure (LVEDP) between 4 and 8 mmHg. The LVEDP increased steadily from 2.7 to 20.6 mmHg within the first 15 minutes of hanging the heart and perfusing it. The diastolic pressure then gradually dropped to 19 mmHg over the next 45 minutes and then remained fairly constant over the remainder of the perfusion duration. This is illustrated in FIG. 28. The error bars represent the standard deviation of the measurements over three different control hearts.

The overall health of the heart was determined by monitoring the developed ventricular pressure. The developed pressure is defined as the difference between the systolic and diastolic pressure. The control hearts developed an average pressure of 77 mmHg during the initial period of perfusion. Thereafter, the average developed pressures started to drop gradually indicating that the hearts were slowly dying from the stress of being removed from the body and artificially perfused. At the end of the 140 minute perfusion period, the developed pressure had dropped from 77 to 30 mmHg. The temperature of the heart and the perfusate were measured at regular intervals using a thermistor based temperature probe (that was built and calibrated in Dr. Valvano's lab) and verified to be 37° C. at all times during the experiment. The average developed pressures in the control hearts are shown in FIG. 29. The error bars represent the standard deviation of the measurements over three different hearts.

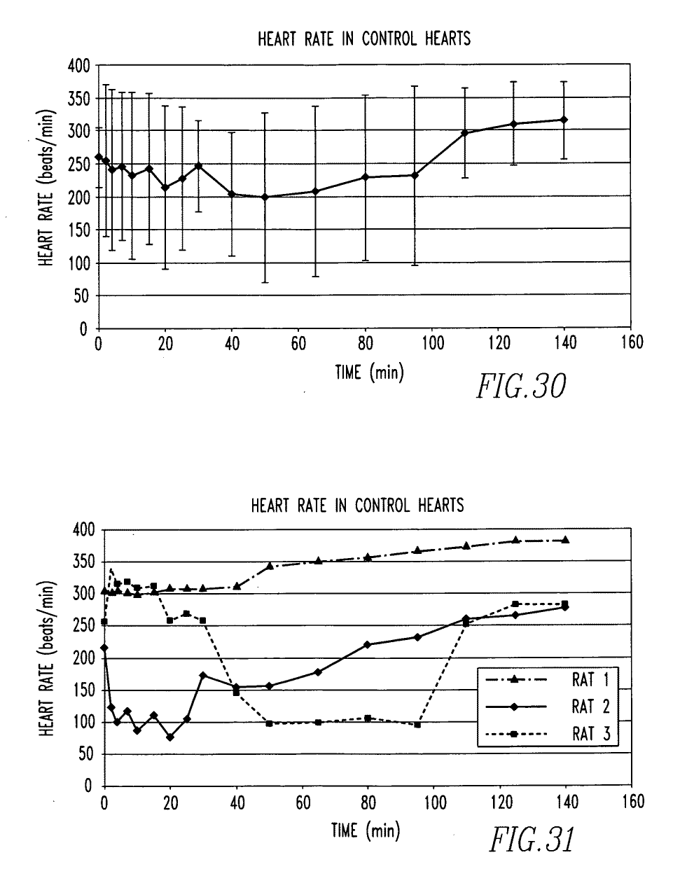

The heart rate was calculated from the LV pressure measurements as an alternative indicator of the overall health of the heart. The control hearts start out with an average heart rate of 260 beats per minute and the rate gradually increases to over 300 beats per minute at the end of the perfusion duration. This instability in the heart rate is because in vivo, the atria and the SA node are not perfused by coronary vessels but by extracardiac vessels, which are severed when the heart is excised for perfusion [22]. Hence the affected tissue is dependent on superfusion by the coronary effluent flowing out of the right atrium for its oxygen, nutrients and temperature control. The variation of the heart rate with time is shown in FIG. 30.

The large standard deviations in the heart rate can be eliminated if the hearts are externally paced. However, external pacing would interfere with the electrical admittance measurements. On examining each of the hearts individually, it is seen that two of the three control hearts exhibit a marked increase in heart rate over the entire perfusion duration. The third heart exhibited an unusual drop in heart rate for 60 minutes during the perfusion interval, the cause of which is unknown. This is illustrated in FIG. 31.

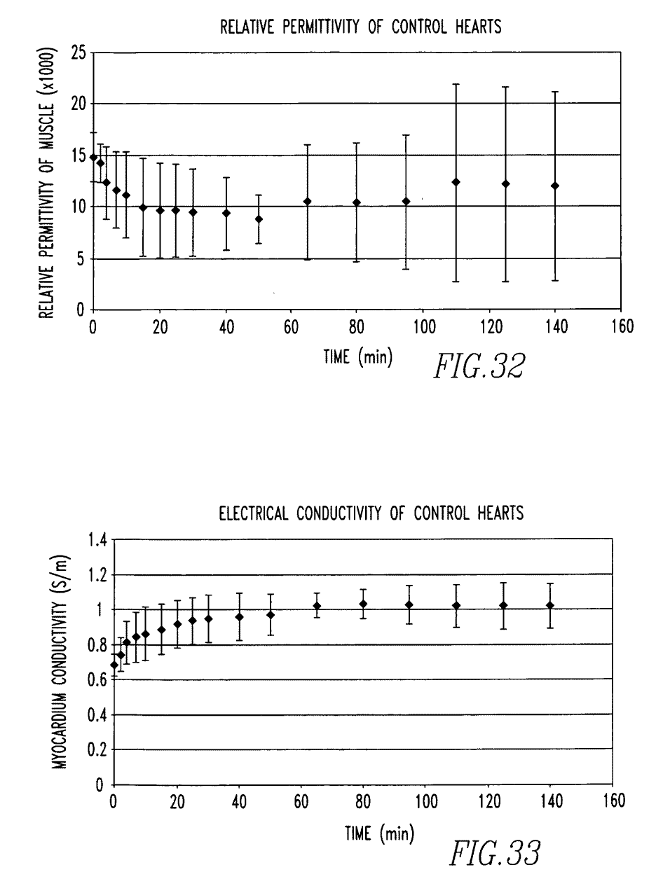

The admittance magnitude |Y|, phase angle θ and left ventricular developed pressure were monitored in real time during the entire 140 minute perfusion duration. The values of |Y| and θ were used to calculate the relative permittivity (∈ᵣ=K.C, after probe capacitance is removed) and conductivity (σ=K.G) of the myocardium based on the measured probe constant K (described by equations 6, 7 and 8). These results are shown in FIGS. 32 and 33.

The average permittivity starts out at 15,000 and decreases to 10,000 over the first 15 minutes after which it remains fairly constant over the next 80 minutes. The average permittivity then rises to 12,500 and remains at that value for the remainder of the perfusion duration.

The average value of myocardial conductivity is 0.75 S/m (7500 μS/cm) when the heart is initially perfused. The conductivity slowly rises to about 1.0 S/m (10000 μS/cm) during the first 50 minutes and remains at that value over the remainder of the perfusion duration. This trend along with the standard deviations is illustrated in FIG. 33.

Samples of the coronary effluent were collected during perfusion to determine the relative amount of LDH release. The samples were analyzed using a commercially available LDH cytotoxicity kit (Cayman Chemicals). The kit consisted of 100 μL each of NAD⁺, Lactic Acid and INT (tetrazolium salt), 15 mL of assay buffer and one vial each of LDH standard and diaphorase. The diaphorase was reconstituted with 150 μL of the assay buffer. Following this, the reaction solution was prepared by mixing 100 μl, each of NAD⁺, Lactic Acid, INT and reconstituted diaphorase with 9.6 mL of assay buffer. When equal amounts of the reaction solution and the coronary effluent samples are mixed together, the following coupled two-step reaction occurs. In the first step, LDH catalyzes the reduction of NAD⁺ to NADH and H⁺ by oxidation of lactate to pyruvate. In the second step of the reaction, diaphorase uses the newly formed NADH and H⁺ to catalyze the reduction of INT to highly colored formazan, which absorbs light of wavelength 490-520 nm. The amount of formazan formed is proportional to the amount of LDH present in the coronary effluent. LDH standard solutions were prepared by adding 1.8 mL of the assay buffer to the vial containing the LDH standard. The concentration of this standard is 200 mU/mL. One unit (U) is the amount of enzyme that catalyzes the reaction of 1 vmol of substrate per minute. This standard solution can be diluted in differing amounts of the assay buffer to obtain desired concentrations of standard solutions. A spectrophotometer was used to determine the absorbance of the effluent samples and the standards at 490 nm. The LDH level in the sample was determined by comparing the absorbance of the sample with the absorbance of the known standards.

The results show that the highest concentration of LDH is present in the samples collected within the first 10 minutes. This is to be expected, since the process of removing the heart from the animal, cannulating the aorta and inserting the ventricular balloon all cause some tissue death. The subsequent samples contain decreasing amounts of this intracellular protein, as seen in Tables 4 and 5. The concentration of LDH in the sample is expressed in terms of μU/ml.

TABLE 4
LDH concentration in control hearts (0 min to 50 min)

| | Time (minutes) | | | | | | | | | | |
|---|---|---|---|---|---|---|---|---|---|---|---|
| Rat # | 0 | 5 | 10 | 15 | 20 | 25 | 30 | 35 | 40 | 45 | 50 |
| 1 | 400 | 138 | 325 | 342 | 396 | 413 | 200 | 129 | 0 | 83 | 46 |
| 2 | 2208 | 1308 | 1300 | 1317 | 1275 | 733 | 717 | 550 | 429 | 308 | 283 |
| 3 | 2367 | 5967 | 7267 | 4408 | 4033 | 3567 | 2925 | 2958 | 2329 | 1700 | 1829 |

TABLE 5
LDH concentration in control hearts (59 min to 144 min)

| | Time (minutes) | | | | | | | | | |
|---|---|---|---|---|---|---|---|---|---|---|
| Rat # | 59 | 64 | 74 | 84 | 94 | 104 | 114 | 124 | 134 | 144 |
| 1 | 33 | 8 | 13 | 13 | 8 | 17 | 4 | 0 | 0 | 4 |
| 2 | 258 | 238 | 217 | 175 | 133 | 163 | 192 | 171 | 150 | 400 |
| 3 | 1958 | 1642 | 1325 | 1117 | 908 | 775 | 642 | 542 | 442 | 425 |

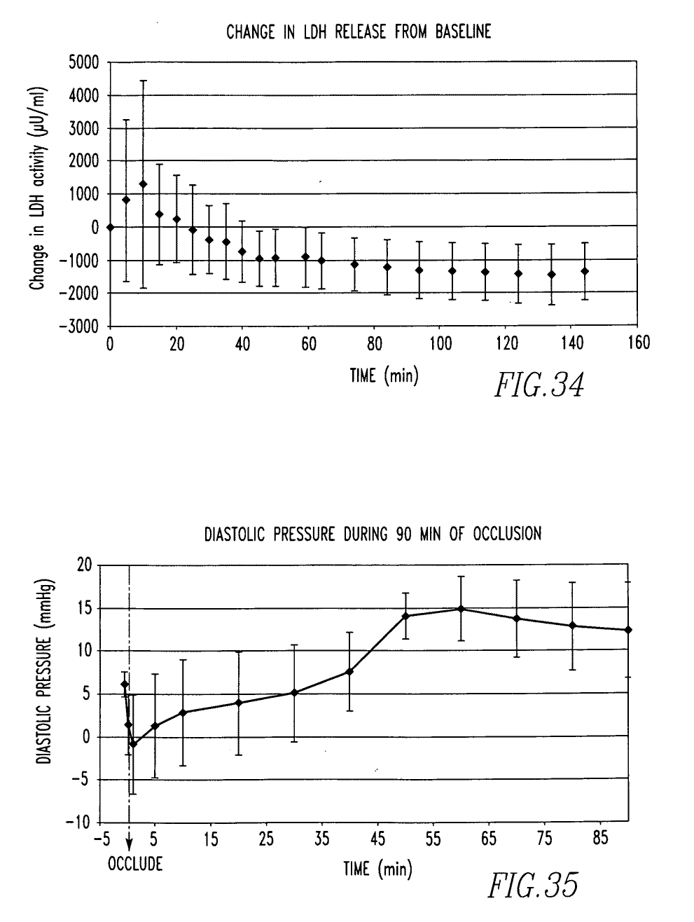

The LDH analysis of the control hearts shows that rat 1 exhibits the least amount of LDH release and should therefore be considered the best control. Rat 3 has a high and sustained LDH release for over 85 minutes and is the worst of the three controls. Rat 3 also exhibited an unusual drop in heart rate for 60 minutes during the perfusion interval, which was illustrated in FIG. 31. The average change in LDH release from baseline (time=0) for the three control hearts is shown in FIG. 34.

Eight adult male WKY rat hearts were subject to 90 min of global no-flow ischemia according to the protocol described in the earlier section. The measured admittance magnitude and phase angle were used to determine the relative permittivity and conductivity of the myocardial tissue during baseline, ischemia and reperfusion. LDH samples were collected during baseline and reperfusion to investigate the incidence of the cell plasma membrane damage due to ischemia.

The LV pressure was monitored in the same manner as in the control heart protocol, by inserting a balloon into the LV. The LVEDP dropped to zero within the first minute of occlusion and then gradually increased to about 5 mmHg over the next 30 min of occlusion. This was followed by a 10-15 min period where the LVEDP increased rapidly from 5 to 15 mmHg, indicating diastolic dysfunction as a result of calcium overload within the cells. The pressure then gradually drops by about 2 mmHg over the next 30 min. On reperfusion, the LVEDP increases to nearly 100 mmHg indicating total diastolic failure.

The measured LVEDP during baseline and the 90 min occlusion period is shown in FIG. 35. The error bars represent the standard deviation of the measurements over seven different hearts.

The admittance magnitude, phase angle and left ventricular developed pressure were monitored in real time during baseline, occlusion and reperfusion. The measured admittance magnitude and phase angles were then used to calculate the relative permittivity (∈ᵣ) and conductivity (σ) of the myocardium. These results are summarized in Tables 6 and 7 for baseline and at specific times during occlusion. The occlusion was performed at time=0 min and all other times on the table are relative to the occlusion time.

TABLE 6
Relative permittivity of myocardium during baseline and 90 min
of occlusion

| | Time (minutes) | | | | | | |
|---|---|---|---|---|---|---|---|
| Rat # | −0.6 | 0.1 | 1.1 | 5 | 10 | 20 | 30 |
| 1 | 13587 | 12555 | 10183 | 10498 | 9396 | 8344 | 8595 |
| 2 | 12263 | 12840 | 13329 | 12950 | 12686 | 12095 | 12102 |
| 3 | 9670 | 9616 | 11461 | 11710 | 11304 | 10943 | 11491 |
| 4 | 3942 | 4047 | 3423 | 4528 | 4849 | 5240 | 6620 |
| 5 | 17317 | 17219 | 14869 | 13621 | 13169 | 12333 | 11627 |
| 6 | 7818 | 6470 | 6068 | 9204 | 8943 | 8450 | 8871 |
| 7 | 11633 | 11374 | 11295 | 9746 | 8751 | 7914 | 7498 |
| Mean | 10890 | 10589 | 10090 | 10322 | 9871 | 9331 | 9543 |
| Std. | 4286 | 4363 | 4027 | 3025 | 2841 | 2574 | 2189 |
| Dev | | | | | | | |

| | Time (minutes) | | | | | |
|---|---|---|---|---|---|---|
| Rat # | 40 | 50 | 60 | 70 | 80 | 90 |
| 1 | 10804 | 13003 | 14229 | 14285 | 13606 | 13061 |
| 2 | 11557 | 13838 | 15543 | 13059 | 11400 | 15409 |
| 3 | 14690 | 13929 | 12393 | 11961 | 12347 | 12103 |
| 4 | 8885 | 10622 | 12974 | 14009 | 14164 | 13908 |
| 5 | 10847 | 11619 | 12445 | 15300 | 16925 | 16468 |
| 6 | 9596 | 11361 | 13182 | 13870 | 13812 | 13634 |
| 7 | 9230 | 12940 | 12765 | 12398 | 12145 | 11599 |
| Mean | 10801 | 12473 | 13362 | 13555 | 13486 | 13740 |
| Std. | 1970 | 1283 | 1142 | 1155 | 1819 | 1732 |
| Dev | | | | | | |

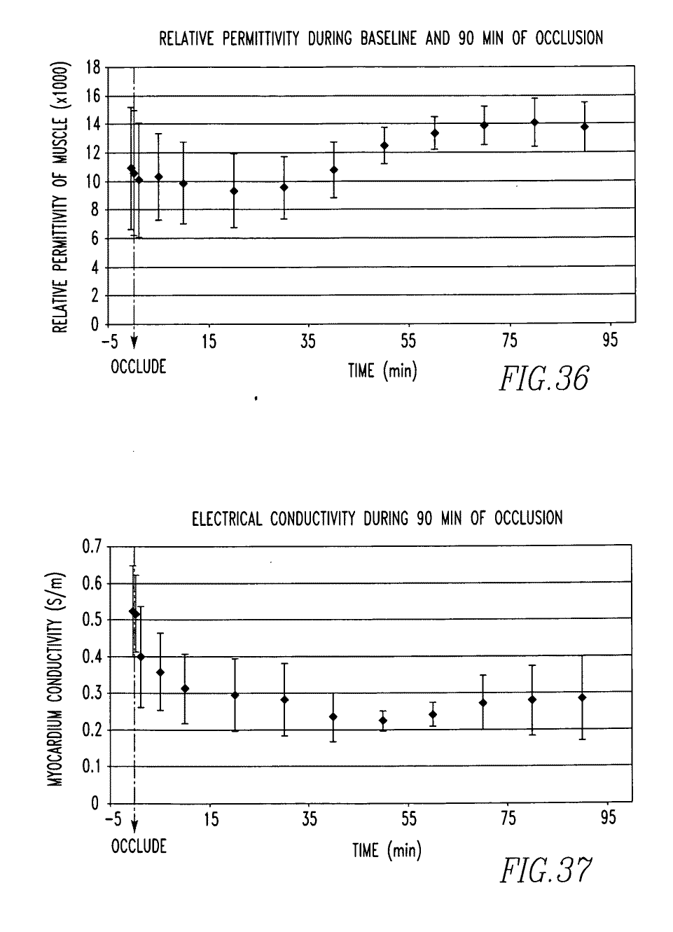

It is seen that the average ∈ᵣ is about 11,000 during baseline. After occlusion, ∈ᵣ drops gradually by about 15% for the first 20 minutes. This is followed by a 10 minute period where ∈ᵣ gradually increases by about 2% following which there is a 30 minute period where ∈ᵣ increases rapidly by nearly 40% to a value of 13,400. After this, the relative permittivity remains fairly constant until reperfusion. This trend along with the standard deviations is illustrated in FIG. 36.

TABLE 7
Electrical conductivity of myocardium during baseline and 90 min
of occlusion

| | Time (minutes) | | | | | | |
|---|---|---|---|---|---|---|---|
| Rat # | −0.6 | 0.1 | 1.1 | 5 | 10 | 20 | 30 |
| 1 | 0.488 | 0.448 | 0.490 | 0.447 | 0.264 | 0.231 | 0.221 |
| 2 | 0.558 | 0.520 | 0.438 | 0.372 | 0.344 | 0.308 | 0.272 |
| 3 | 0.552 | 0.491 | 0.366 | 0.292 | 0.269 | 0.246 | 0.223 |
| 4 | 0.711 | 0.667 | 0.623 | 0.509 | 0.470 | 0.451 | 0.441 |
| 5 | 0.394 | 0.404 | 0.396 | 0.396 | 0.379 | 0.382 | 0.382 |
| 6 | 0.608 | 0.656 | 0.222 | 0.307 | 0.293 | 0.291 | 0.289 |
| 7 | 0.348 | 0.435 | 0.256 | 0.190 | 0.173 | 0.159 | 0.153 |
| Mean | 0.523 | 0.517 | 0.399 | 0.359 | 0.313 | 0.296 | 0.283 |
| Std. | 0.125 | 0.105 | 0.137 | 0.106 | 0.095 | 0.097 | 0.099 |
| Dev | | | | | | | |

| | Time (minutes) | | | | | |
|---|---|---|---|---|---|---|
| Rat # | 40 | 50 | 60 | 70 | 80 | 90 |
| 1 | 0.217 | 0.222 | 0.227 | 0.233 | 0.228 | 0.224 |
| 2 | 0.246 | 0.238 | 0.235 | 0.265 | 0.313 | 0.235 |
| 3 | 0.194 | 0.198 | 0.228 | 0.271 | 0.256 | 0.245 |
| 4 | 0.231 | 0.228 | 0.284 | 0.312 | 0.321 | 0.323 |
| 5 | 0.367 | 0.275 | 0.288 | 0.423 | 0.479 | 0.531 |
| 6 | 0.240 | 0.224 | 0.226 | 0.227 | 0.228 | 0.232 |
| 7 | 0.151 | 0.190 | 0.205 | 0.213 | 0.213 | 0.205 |
| Mean | 0.235 | 0.225 | 0.242 | 0.278 | 0.291 | 0.285 |
| Std. | 0.067 | 0.028 | 0.032 | 0.072 | 0.093 | 0.115 |
| Dev | | | | | | |

The average value of myocardial conductivity is 0.52 S/m during baseline. The conductivity drops rapidly to 0.4 S/m during the first minute of occlusion and then reduces further to 0.3 S/m over the next 10 minutes. Following this, the conductivity drop is more gradual and falls to 0.23 S/m over the next 40 minutes and then slowly increases to 0.29 S/m during the remainder of the occlusion period. This trend along with the standard deviations is illustrated in FIG. 37.

The reduction in myocardial conductivity during occlusion is due to the absence of the high conductivity perfusate in the myocardium. On reperfusion, the conductivity increases almost instantaneously to 0.7 S/m and remains elevated during the entire 20 min reperfusion period. This elevated conductivity value was seen in the three control hearts as well and was shown in FIG. 33. The conductivity of the perfusate was measured to be 1.2 S/m.

Samples of the coronary effluent were collected during baseline and reperfusion to determine the amount of LDH release. The samples were analyzed as described above. The results show that the highest concentration of LDH is present in the sample collected immediately after reperfusion. This is to be expected, since almost all the LDH released during the 90 min occlusion period remains in the extracellular space and will be washed out all at once when reperfusion begins. The subsequent samples contain decreasing amounts of this intracellular protein, as is evident from Table 8. The concentration of LDH in the sample is expressed in terms of μU/ml. One unit (U) is the amount of enzyme that catalyzes the reaction of 1 μmol of substrate per minute.

TABLE 8
LDH concentration in hearts subject to 90 min of ischemia

| | Sample # | | | | |
|---|---|---|---|---|---|
| Rat # | 1 | 2 | 3 | 4 | 5 |
| 1 | 4300 | 1633 | 21600 | 6033 | 4983 |
| 2 | 550 | 23733 | 12417 | 8767 | 5367 |
| 3 | 2483 | 22967 | 5167 | 2400 | 1250 |
| 4 | 2017 | 26017 | 10767 | 7017 | 5983 |
| 5 | 2500 | 28950 | 10150 | 6717 | 6233 |
| 6 | 6783 | 39317 | 16300 | 12167 | 10250 |
| 7 | 12717 | 50800 | 17733 | 16100 | 14483 |

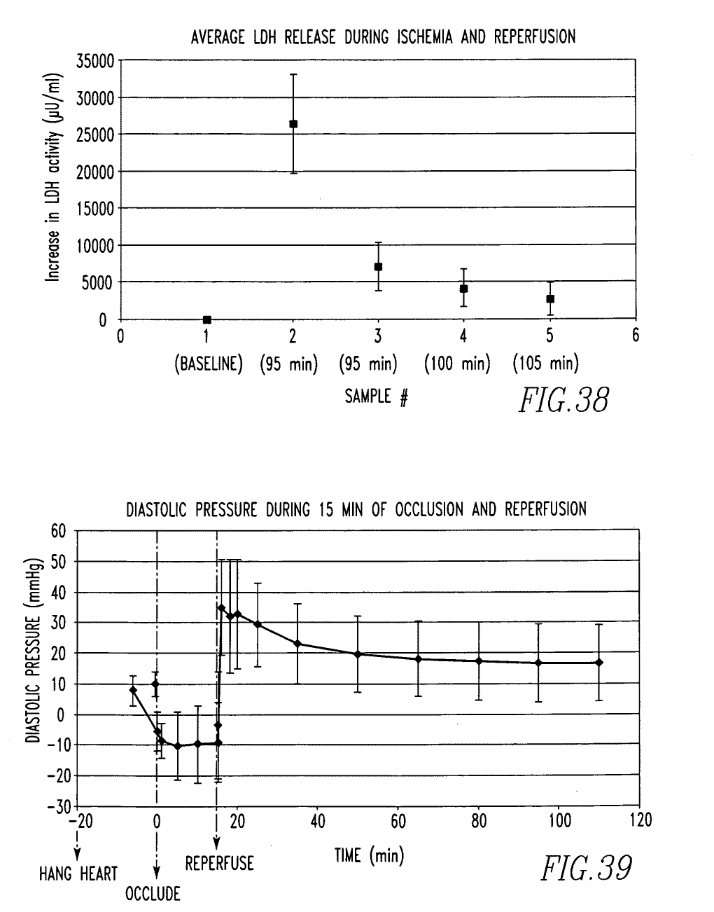

Sample 1 is taken at baseline and sample 2 is taken immediately upon reperfusion. Samples 3, 4 and 5 are taken after subsequent 5 min intervals. The average increase in LDH release from baseline for all seven hearts is plotted in FIG. 38. The error bars represent the standard deviation of the measurements.

Eight adult male WKY rat hearts were subject to 15 min of global no-flow ischemia followed by 95 min of reperfusion according to the protocol described in the earlier section. The measured admittance magnitude and phase angle were used to determine the relative permittivity and conductivity of the myocardial tissue during baseline, ischemia and reperfusion. LDH samples were collected during baseline and reperfusion to investigate the incidence of the cell plasma membrane damage due to ischemia. Out of the eight rat hearts subject to this protocol, two hearts did not recover from the occlusion and did not develop measurable ventricular pressures over the entire reperfusion period. Data from these two hearts were excluded from the data analysis.

The LV pressure was monitored in the same manner as in the control hearts protocol, by inserting a balloon into the LV. The LVEDP dropped rapidly within the first minute of occlusion and remained at −10 mmHg over the entire 15 min occlusion period. On reperfusion, the LVEDP increased to 35 mmHg within a minute and then gradually dropped to 16 mmHg over the entire 95 min reperfusion period. This is illustrated in FIG. 39. The error bars represent the standard deviation of the measurements over six different hearts.

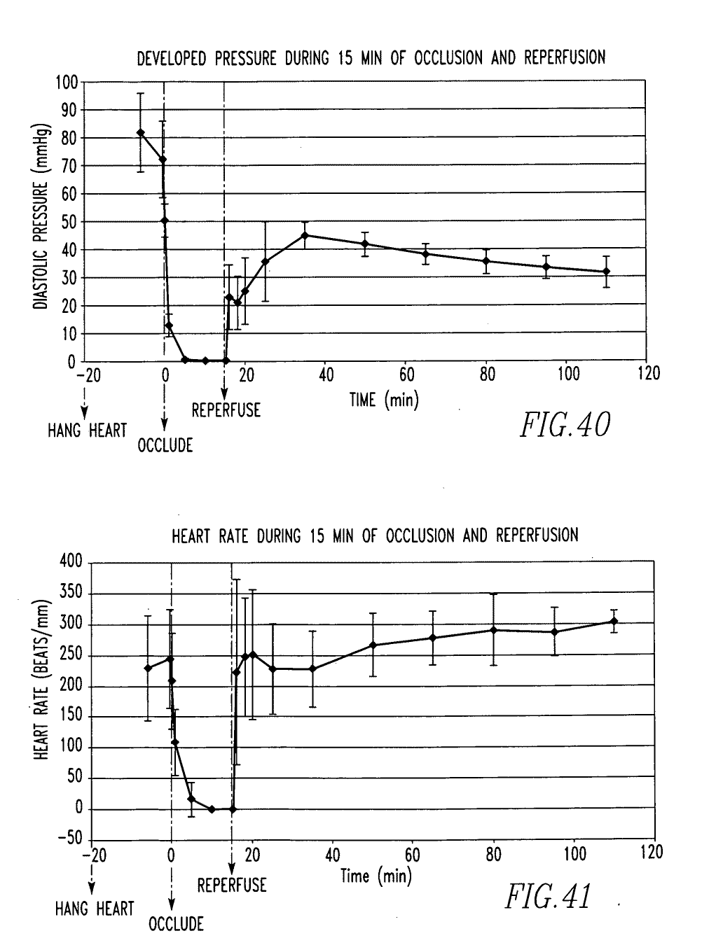

On reperfusion, six of the eight hearts recovered and were able to achieve developed pressures of 45 mmHg after 20 minutes of reperfusion. Thereafter, the developed pressures started to drop gradually indicating that the hearts were slowly dying. At the end of the 95 min reperfusion period, the developed pressure had dropped to 31 mmHg. This is illustrated in FIG. 40. The error bars represent the standard deviation of the measurements over six different hearts.

The heart rate was calculated from the LV pressure measurements to further ensure that the hearts had recovered from ischemia during the reperfusion period. It is seen that the heart stops beating completely during occlusion. Although there is a large standard deviation in the measured heart rate within the first 10 minutes of reperfusion, the rate stabilizes to about 275 beats per minute after 35 minutes of reperfusion. This is shown in FIG. 41.

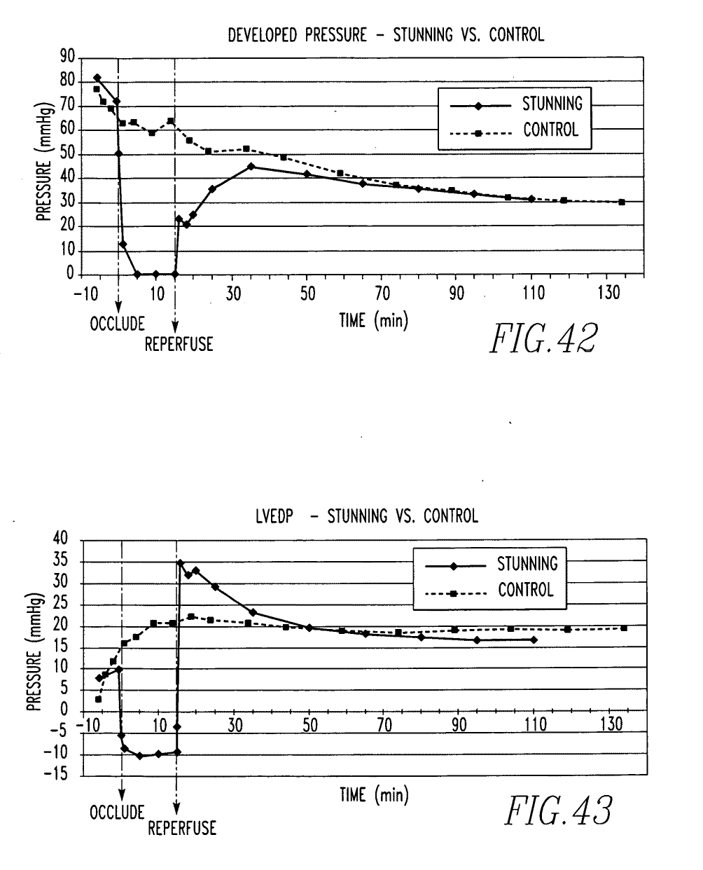

As previously described, stunning is the mechanical dysfunction that persists after reperfusion despite the absence of irreversible damage and despite restoration of normal coronary flow. The developed ventricular pressures in these six hearts were compared with the pressures developed in the controls to verify that true stunning had occurred. The left ventricular end diastolic pressures (LVEDP) of the stunned hearts were also compared with the controls. These are shown in FIGS. 42 and 43, which confirm that the hearts subject to 15 minutes of occlusion eventually recover and attain diastolic and developed pressures identical to the controls. It is also evident from these Figures that the stunned hearts recover within 35 min of reperfusion.

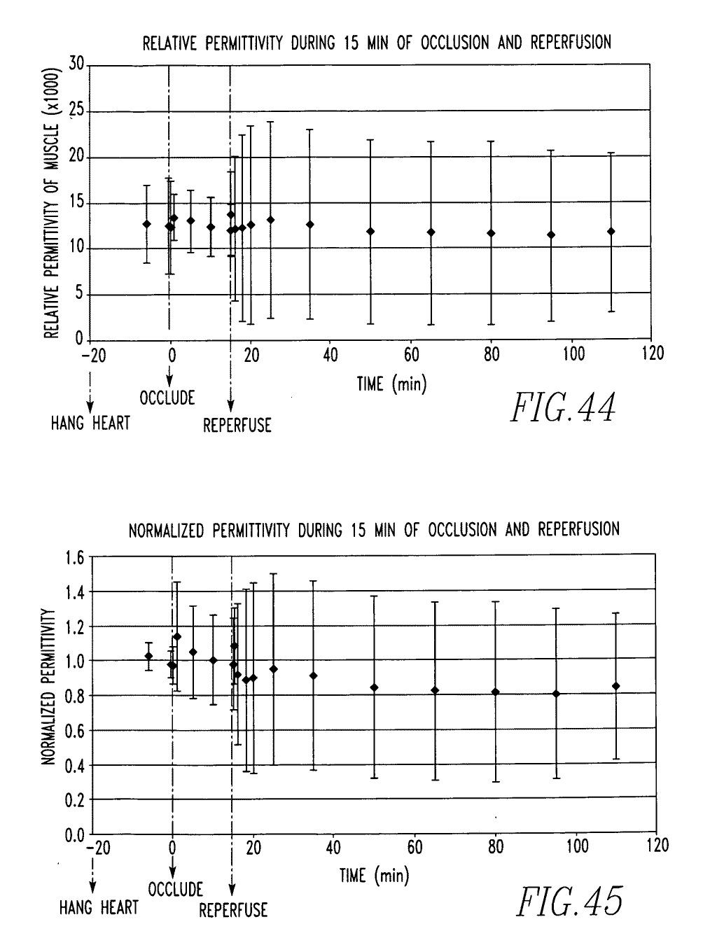

As in the ischemia protocol, the admittance magnitude, phase angle and left ventricular developed pressure were monitored in real time during baseline, occlusion and reperfusion and used to calculate the relative permittivity (∈ᵣ) and conductivity (σ) of the myocardium. These results are shown in FIGS. 44, 45 and 46 for baseline and at specific times during occlusion and reperfusion. The occlusion was performed at time=0 min and all other times on the graph are relative to the occlusion time.

The error bars represent the standard deviation of the measurements over six different hearts. The permittivity data do not show any statistically significant changes during baseline, occlusion and subsequent reperfusion. The normalized permittivity values do not show any statistically significant trends either (FIG. 45). This indicates that the relative permittivity of the myocardium may not be a good indicator of myocardial stunning, and is more useful as an indicator of ischemia and diastolic dysfunction.

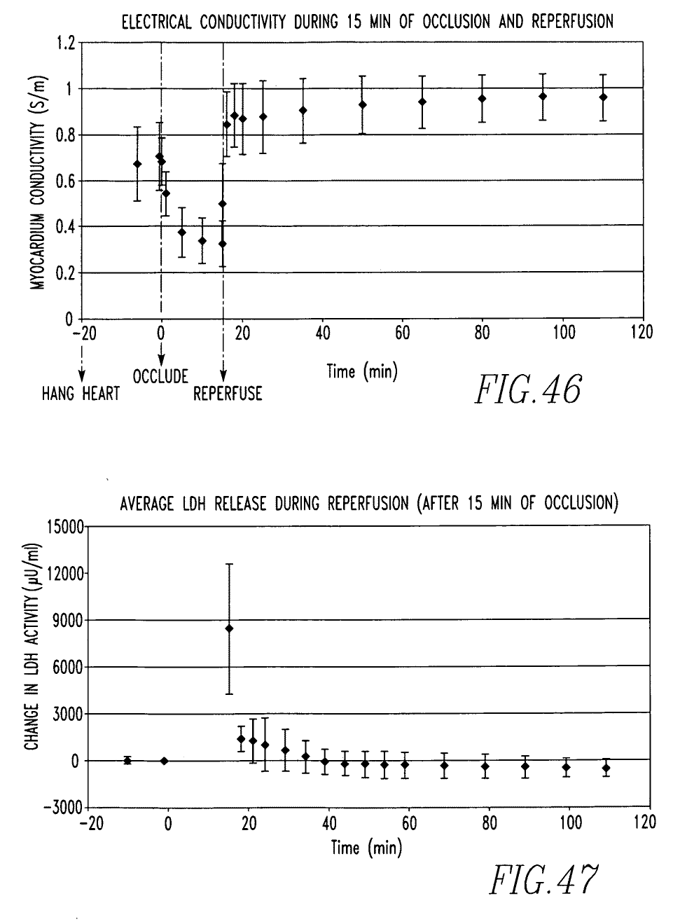

The average value of myocardial conductivity is 0.67 S/m during baseline. The conductivity drops rapidly to 0.54 S/m during the first minute of occlusion and then reduces further to 0.33 S/m over the next 14 minutes. On reperfusion, the conductivity increases almost instantaneously to 0.84 S/m and remains elevated during the entire 95 min reperfusion period. This trend along with the standard deviations is illustrated in FIG. 46.

The reduction in myocardial conductivity during occlusion is due to the absence of the high conductivity perfusate in the myocardium. This trend is similar to that observed in hearts subject to the 90 min ischemia protocol. On reperfusion, the conductivity increases almost instantaneously and reaches a maximum value of 0.96 S/m during the reperfusion period. This elevated conductivity value was seen in the three control hearts as well, and has been shown in FIG. 33.

Samples of the coronary effluent were collected during baseline and reperfusion to determine the amount of LDH release. Two samples were collected at baseline; one immediately after the heart was hung on the system and instrumented and the second one after ten minutes of stabilization. The heart was then subjected to 15 min of global no-flow ischemia, followed by 95 min of reperfusion. The third sample was collected immediately upon reperfusion and three more samples were collected at 3 min intervals. Seven subsequent samples were collected at 5 min intervals and the last five samples were collected at 10 min intervals. The samples were analyzed using the Cayman LDH cytotoxicity kit. The results indicate the highest concentration of LDH in the sample collected immediately after reperfusion, since almost all the LDH released during the 15 min occlusion period remains in the extracellular space and will be washed out all at once when reperfusion begins. This LDH concentration is lower than that obtained after 90 min of occlusion, which implies that there is substantially less changes in cell membrane permeability for shorter occlusion periods. The subsequent samples contain reducing amounts of this intracellular protein, as is evident from Tables 9 and 10.

TABLE 9
LDH concentration in hearts subject to 15 min of ischemia (baseline
and partial reperfusion)

| | Time (min) | | | | | | | | |
|---|---|---|---|---|---|---|---|---|---|
| Rat # | −10 | −1 | 15 | 18 | 21 | 24 | 29 | 34 | 39 |
| 1 | 533 | 770 | 12870 | 2708 | 2333 | 1183 | 795 | 395 | 270 |
| 2 | 1445 | 908 | 26565 | 2745 | 1083 | 933 | 1008 | 445 | 345 |
| 3 | 1118 | 883 | 15675 | 1703 | 893 | 403 | 538 | 363 | 63 |
| 4 | 558 | 783 | 10895 | 1508 | 1320 | 1045 | 1126 | 1208 | 908 |
| 5 | 420 | 445 | 7070 | 695 | 658 | 2045 | 1501 | 958 | 801 |
| 6 | 1070 | 1008 | 4420 | 3083 | 5108 | 5520 | 4453 | 3387 | 2320 |
| 7 | 1683 | 1758 | 9820 | 3858 | 2533 | 1458 | 1224 | 991 | 758 |
| 8 | 942 | 525 | 4550 | 2450 | 2142 | 1683 | 1125 | 567 | 500 |

TABLE 10
LDH concentration in hearts subject to 15 min of ischemia
(remainder of reperfusion)

| | Time (min) | | | | | | | | |
|---|---|---|---|---|---|---|---|---|---|
| Rat # | 44 | 49 | 54 | 59 | 69 | 79 | 89 | 99 | 109 |
| 1 | 201 | 133 | 66 | 0 | 0 | 0 | 0 | 0 | 0 |
| 2 | 133 | 183 | 20 | 8 | 45 | 0 | 0 | 0 | 0 |
| 3 | 113 | 0 | 13 | 53 | 3 | 0 | 0 | 0 | 0 |
| 4 | 608 | 641 | 674 | 708 | 683 | 658 | 633 | 608 | 583 |
| 5 | 645 | 595 | 545 | 495 | 445 | 391 | 337 | 283 | 229 |
| 6 | 2337 | 2353 | 2370 | 2245 | 2120 | 1995 | 1799 | 1603 | 1408 |
| 7 | 658 | 558 | 458 | 424 | 391 | 358 | 374 | 391 | 408 |
| 8 | 433 | 358 | 283 | 208 | 133 | 100 | 67 | 33 | 0 |

The concentration of LDH in the sample is expressed in terms of μc U/ml. One unit (U) is the amount of enzyme that catalyzes the reaction of 1 μmol of substrate per minute.

The times represented in Tables 9 and 10 correspond to the time following the start of occlusion. Thus, the occlusion is started at time=0 min and reperfusion begins at time=15 min. The two baseline values are represented by time=−10 min and −1 min.

Rat #4 and #5 did not recover from the 15 min occlusion and were excluded from the analysis. An abnormally high level of LDH release was observed in rat 2 immediately upon reperfusion, indicating a high degree of cell necrosis. The average increase in LDH release from baseline for the remaining five hearts is plotted in FIG. 47. The error bars represent the standard deviation of the measurements over five different hearts.

All the LDH release occurred within 20 minutes reperfusion, indicating that cell necrosis only occurred during the occlusion period and no further cell necrosis occurs during the recovery period.

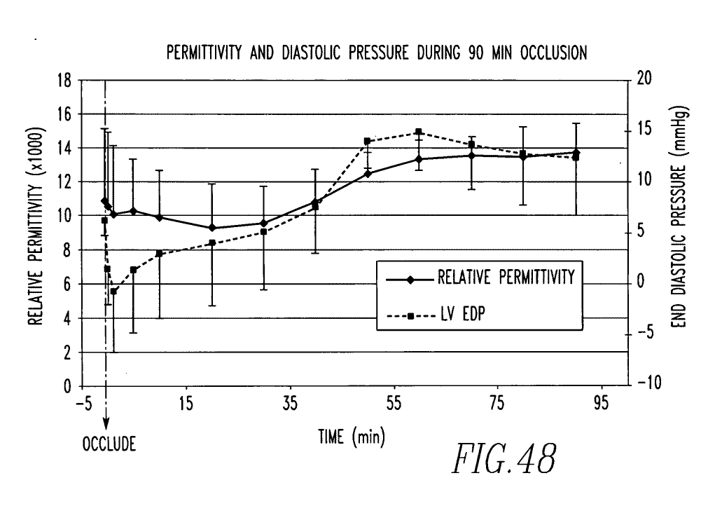

The myocardial ischemia studies confirm the occurrence of diastolic dysfunction during a prolonged period of no-flow ischemia. This is characterized by a rapid increase in diastolic pressure within the left ventricle and occurs about 30 minutes after the onset of ischemia. An interesting observation here is the behavior of the myocardial permittivity during this period. The permittivity reduces during the first 20 min following occlusion and then starts to increase. This inflection point is observed prior to the onset of diastolic dysfunction and precedes it by 15 minutes. This is illustrated in FIG. 48.

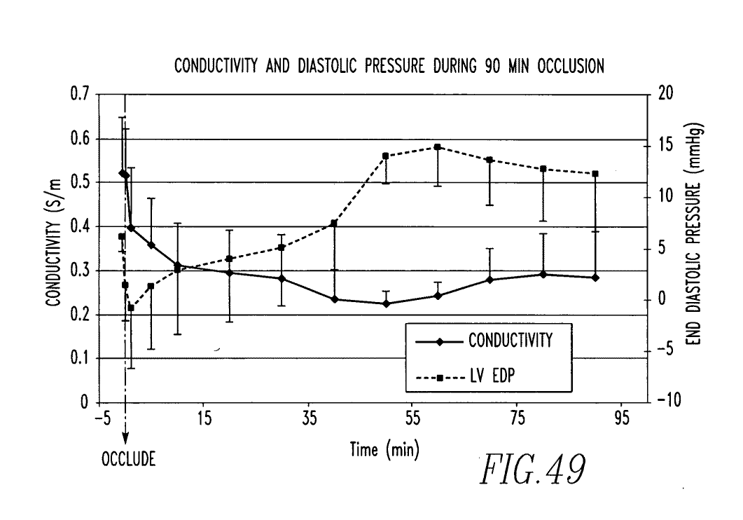

Intracellular calcium levels have been reported to be elevated during ischemia [32, 36, 53] and remain elevated until reperfusion. The elevated calcium levels are due to the reduced rate of calcium uptake by the sarcoplasmic reticulum. This excess calcium binds Troponin I to actin in thin myofilaments to hold the troponin-tropomyosin complex in place. This prevents ventricular relaxation and results in stiffening of the ventricular muscle. As a result, diastolic pressure increases leading to diastolic dysfunction. Diastolic dysfunction is hence caused by calcium overload in the cytoplasm due to a breakdown of the functioning of the sarcoplasmic reticulum. This alteration of the intracellular electrolyte composition increases the trans-membrane charge distribution across the cell membrane and causes a sudden increase in permittivity. The myocardial conductivity, however, exhibits no such trends and exhibits no correlation with diastolic dysfunction. FIG. 49 shows a comparison between myocardial conductivity and diastolic pressure during baseline and 90 min of occlusion.

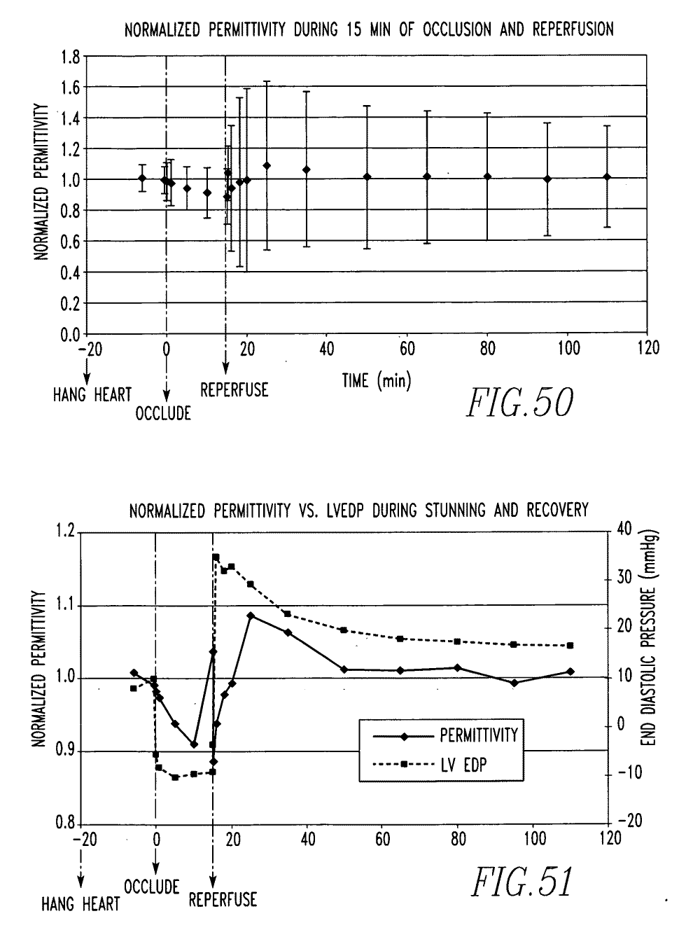

Eight rats were subject to the myocardial stunning protocol. Only six of the eight hearts recovered on reperfusion and these hearts were analyzed for trends in permittivity and conductivity during occlusion and reperfusion. The permittivity results did not show any statistically significant trends during baseline, occlusion and subsequent reperfusion. Out of these six hearts, one of the hearts exhibited abnormally high LDH levels and another heart had permittivity values gradually reduce to zero on reperfusion. These two hearts were also excluded from the analysis and the average relative permittivity of the four remaining hearts is plotted in FIG. 50.

The normalized permittivity results indicate a gradual decrease in permittivity from baseline during the 15 min occlusion period. The elevated LDH activity that has been observed during this period indicates increasing plasma membrane permeability due to necrosis. As a result, the membrane becomes more permeable to Na⁺, K⁺, Ca²⁺ and other ion species. This affects the trans-membrane charge distribution that is responsible for the physical origin of permittivity. Since the permittivity is seen to gradually decrease, it can be concluded that the trans-membrane charge density decreases during the initial 15 min of ischemia. The permittivity increases rapidly upon reperfusion and remains elevated above baseline values for about 35 min following reperfusion. The elevated permittivity could be an indicator of stunned myocardium, since it was shown above that the stunned hearts recover after 35 min of reperfusion. FIG. 51 shows a comparison between normalized mean permittivity and LVEDP during baseline, 15 min of occlusion and 95 min of reperfusion (n=4).

Stunning is characterized by an elevation in end diastolic pressure, possibly due to an overload of Ca²⁺ ions in the cytoplasm. The Ca²⁺ levels remain elevated during the early phase of reperfusion. A specific hypothesis regarding this has been suggested by Grinwald [21]. There is an accumulation of intracellular sodium during ischemia due to the inhibition of Na⁺/K⁺ -ATPase. The Na⁺/Ca²⁺ exchanger also remains suppressed by acidosis. Increased Na⁺/H⁺ exchange due to accumulation of H⁺ during ischemia also favors sodium overload. On reperfusion, the rapid reversal of acidosis allows the reactivation of the Na⁺/Ca²⁺ exchanger when sodium overload has not been resolved. The enhanced sodium efflux causes movement of calcium into the cells, causing the Ca²⁺ levels to remain elevated during the early phase of reperfusion.

This elevation in intracellular Ca²⁺ along with a gradual restoration of membrane permeability to normal levels could cause an increase in myocardial permittivity (similar to that observed during the 90 min ischemia protocol). Once the functioning of the sarcoplasmic reticulum and other calcium transport pumps is restored, the Ca²⁺ levels in the cells return to normal and the diastolic pressures reduce to normal values. The permittivity should hence return to baseline, thereby indicating recovery from stunning. This is in contrast with the ischemic hearts where the hearts do not recover once diastolic dysfunction has set in.

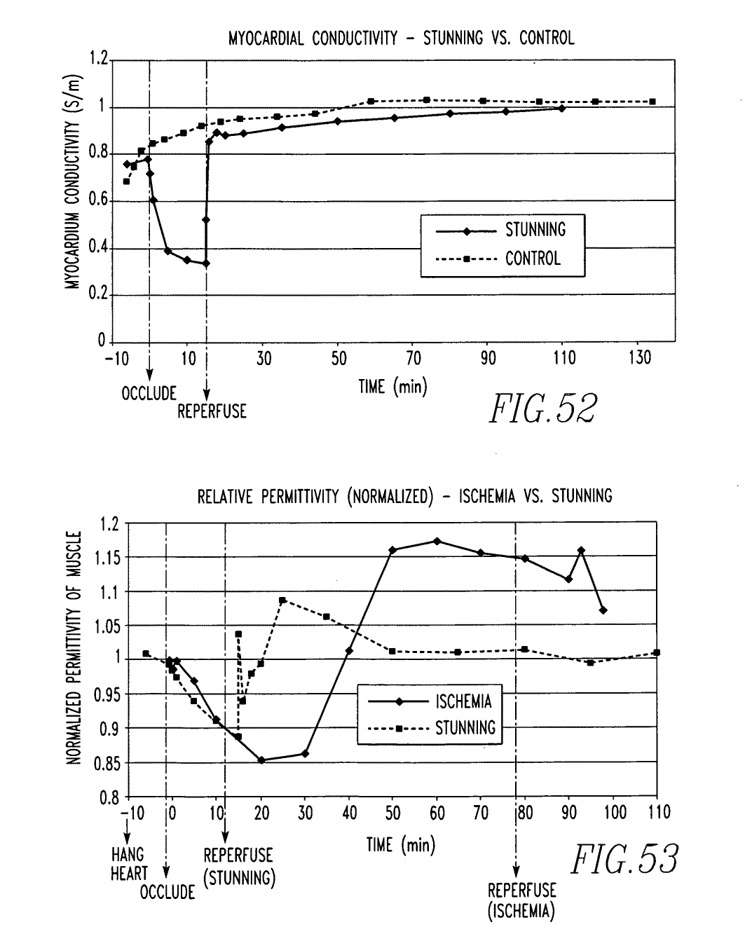

The myocardial conductivity in the four selected stunned hearts was compared with the controls and is shown in FIG. 52. The occlusion causes a sudden drop in myocardial conductivity due to the absence of the high conductivity perfusion fluid. On reperfusion, the conductivity returns to normal value almost instantly and is not affected by the stunned state of the myocardium.

From the above graph, it is evident that myocardial conductivity cannot be used as an indicator of stunning in the myocardium. It may be useful in the detection of no-flow ischemia, since the cessation of blood flow will cause a rapid decrease in the conductivity of the tissue.

The measured permittivity and conductivity during ischemia and stunning has been described in detail above. The permittivity and conductivity values were normalized with respect to baseline values to enable comparison between ischemia and stunning. Normalizing with respect to baseline also causes each heart to serve as its own control, thereby eliminating differences that arise among different hearts as a result of baseline variations. FIG. 53 shows a normalized relative permittivity—ischemia vs. stunning. FIG. 54 shows a normalized conductivity—ischemia vs. stunning.

In summary, an instrument (RamsES I) was designed and developed to determine the magnitude and phase angle of the electrical admittance of tissue in real time. The system was tested on isolated rat heart models of stunning and ischemia. The key findings have been listed below and summarized in Table 11.
Myocardial permittivity decreases during the first 20 min of global no flow ischemia.
Prolonged ischemia (30 min in the isolated rat heart) eventually causes diastolic dysfunction, verified by an increase in LVEDP.
The permittivity increases prior to the onset of diastolic dysfunction in the left ventricle.
Reperfusion after 15 min of occlusion results in myocardial stunning, with the tissue recovering after 35 min of reperfusion.
The permittivity is temporarily elevated in the stunned myocardium and returns to baseline on recovery from stunning.
The myocardial conductivity decreases drastically during occlusion and returns back to baseline almost instantaneously upon reperfusion.
The conductivity measurements are dominated by the high conductivity perfusate in the myocardium and are not affected by the state of the myocardium.
Increase in myocardial permittivity is as good an indicator of diastolic dysfunction as an increase in diastolic pressure.

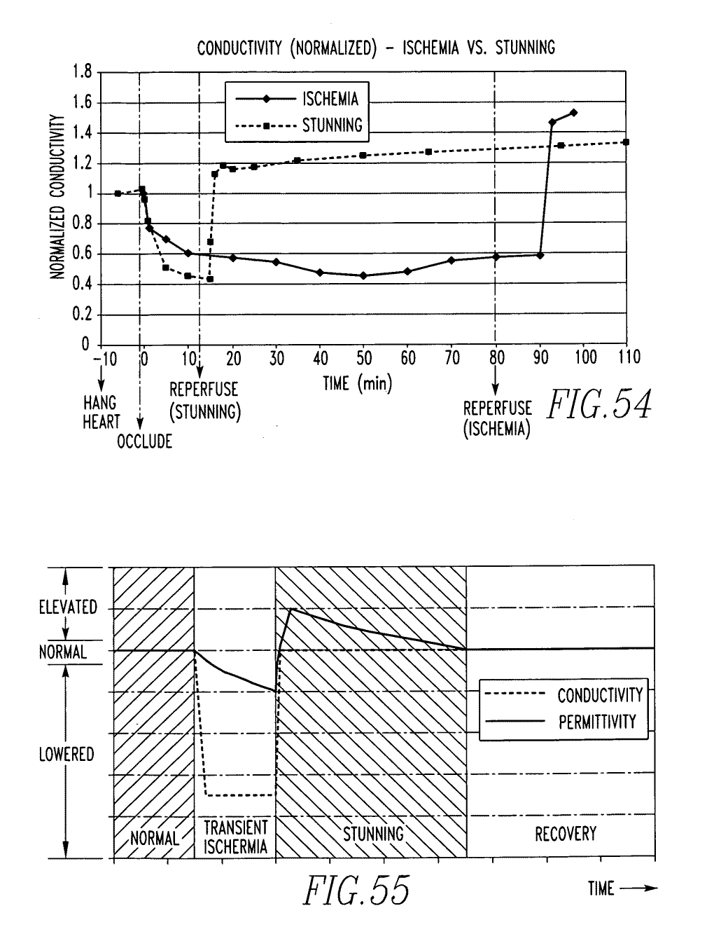

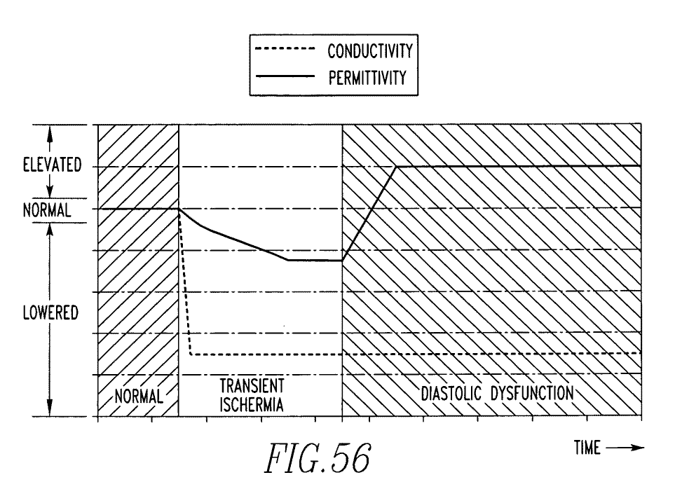

The effects of myocardial ischemia, stunning and diastolic dysfunction on the permittivity, conductivity and LVEDP are summarized in FIGS. 55 and 56 as well as in Table 11. The relative permittivity of the myocardium is seen to decrease by 15% from baseline during 20 min of global no flow ischemia until the onset of diastolic dysfunction. The conductivity is also decreased due to the absence of the high conductivity perfusate (blood) in the myocardium. The onset of diastolic dysfunction is seen to increase the permittivity to 18% above the baseline values. Reperfusion before the onset of diastolic dysfunction causes the permittivity to rise to 10% above baseline (stunned myocardium) and gradually decrease towards baseline as the heart recovers from stunning. The conductivity returns to baseline almost instantaneously upon reperfusion.

TABLE 11
Summary of electrical properties and LV pressure during
ischemia and stunning

| | Permittivity | Conductivity | LVEDP |
|---|---|---|---|
| Ischemia | decreases | decreases | decreases |
| Stunning | increases | unchanged | increases |
| Diastolic dysfunction | increases | decreases | increases |

The trans-membrane charge density is responsible for the physical origin of myocardial permittivity. During global no flow ischemia, cell necrosis occurs and is accompanied by mitochondrial swelling and increased plasma membrane permeability [6, 7, 52, 59]. This is manifested by elevated levels of LDH in the extracellular space. As a result, the membrane may become more permeable to several ion species like Na⁺, K⁺ and Ca²⁺. This would reduce the trans-membrane charge distribution, thereby lowering the measured tissue permittivity. Diastolic dysfunction is characterized by a rapid increase in LVEDP due to calcium overload in the cytoplasm (due to breakdown in the functioning of the sarcoplasmic reticulum [6, 7, 52]). This alteration of the intracellular electrolyte composition increases the trans-membrane charge distribution across the cell membrane and would explain the sudden increase in permittivity. Myocardial stunning also causes an intracellular calcium overload, primarily due to the reactivation of the Na⁺/Ca²⁺ exchanger when the intracellular sodium overload has not been resolved. Since coronary perfusion has been restored in stunned myocardium, the functioning of voltage gated membrane ion channels is restored and membrane permeability gradually returns to normal. All these mechanisms cause rapid changes in the intracellular and extracellular ion concentrations and affect the charge distribution across the cell membranes. This could explain the increased myocardial permittivity during stunning.

References, all of which are incorporated by reference herein.
1. Arthur, W. and Kaye, G. C. Clinical use of intracardiac impedance: current applications and future perspectives. PACE 24[Pt. I]:500-506, 2001.
2. Bolli, R. Mechanism of myocardial “stunning”. Circulation. 82:723-738, 1990.
3. Bolli, R. and Marban, E. Molecular and cellular mechanisms of myocardial stunning. Physiol. Rev. 79:609-634, 1999.
4. Cascio, W. E., Yan, G. X. and Kleber, A. G. Passive electrical properties, mechanical activity, and extracellular potassium in arterially perfused and ischemic rabbit ventricular muscle. Circ. Res. 66:1461-1473, 1990.
5. Cinca, J., Warren, M., Carreno, A., Tresanchez, M., Armadans, L., Gomez, P. and Soler-Soler, J. Changes in myocardial electrical impedance induced by coronary artery occlusion in pigs with and without preconditioning. Circulation. 96:3079-3086, 1997.
6. Cinca, J., Warren, M., Rodriguez-Sinovas, A., Tresanchez, M., Carreno, A., Bragos, R., Casas, O., Domingo, A. and Soler-Soler, J. Passive transmission of ST segment changes in low electrical resistance myocardial infarct scar in the pig. Cardiovasc. Res. 40:103-112, 1998.
7. Dekker, L. R., Fiolet, J. W., VanBavel, E., Coronel, R., Opthof, T., Spaan, J. A. and Janse, M. J. Intracellular Ca²⁺, intercellular electrical coupling, and mechanical activity in ischemic rabbit papillary muscle. Effects of preconditioning and metabolic blockade. Circ. Res. 79:237-246, 1996.
8. del Rio, C. L., McConnell, P. I., Clymer, B. D., Dzwonczyk, R., Michler, R. E., Billman, G. E. and Howie, M. B. Early time course of myocardial electrical impedance during acute coronary artery occlusion in pigs, dogs, and humans. J. Appl. Physiol. 99(4):1576-1581, 2005.
9. Ellenby, M. I., Small, K. W., Wells, R. M., Hoyt, D. J. and Lowe, J. E. On-line detection of reversible myocardial ischemic injury by measurement of myocardial electrical impedance. Ann. Thorac. Surg. 44:587-597, 1987.
10. Fallert, M. A., Mirotznik, M. S., Downing, S. W., Savage, E. B., Foster, K. R., Josephson, M. E. and Bogen, D. K. Myocardial electrical impedance mapping of ischemic sheep hearts and healing aneurysms. Circulation. 87:199-207, 1993.
11. Feldman, M. D., Yi Mao, Valvano, J. W., Pearce, J. A. and Freeman, G, L. Development of a multi frequency conductance catheter based system to determine LV function in mice. Am. J. Physiol. Heart. Circ. Physiol. 279:H1411-H1420, 2000.
12. Fernandez, D. J. Frequency scalable left ventricular admittance measuring instrument for determining cardiac function. Thesis (M.S. in Engineering)—University of Texas at Austin, 2006.
13. Flores, J., DiBona, D. R., Beck, C. H. and Leaf, A. The role of cell swelling in ischemic renal damage and the protective effect of hypertonic solute. J. Clin. Invest. 51:118-126, 1972.
14. Foster, K. S., Petit, R. L., Pearce, J. A., Bourland, J. D. and Geddes, L. A. Tissue resistivity measurement using an annular tetrapolar electrode. J. Clin. Eng. 6:208-212, 1981.
15. Frank, A., Rauen, U. and de Groot, H. Protection by glycine against hypoxic injury of rat hepatocytes: inhibition of ion fluxes through non-specific leaks. J. Hepatol. 32:58-66, 2000.
16. Gabriel, C., Gabriel, S, and Corthout, E. The dielectric properties of biological tissues: I. Literature survey. Phys. Med. Biol. 41:2231-2250, 1996.
17. Gabriel, S., Lau, R. W. and Gabriel, C. The dielectric properties of biological tissues. II. Measurements in the frequency range 10 Hz to 20 GHz. Phys. Med. Biol. 41:2251-2269, 1996.
18. Gabriel, S., Lau, R. W. and Gabriel, C. The dielectric properties of biological tissues. III. Parametric models for the dielectric spectrum of tissues. Phys. Med. Biol. 41:2270-2282, 1996.
19. Gebhard, M. M., Gersing, E., Brockhoff, C. J., Schnabel, P. A. and Bretschneider, H. J. Impedance spectroscopy: A method for surveillance of ischemic tolerance of the heart. Thorac. Cardiovasc. Surg. 35:26-32, 1987.
20. Gores, G. J., Nieminen, A. L., Wray, B. E., Herman, B. and Lemasters, J. J. Intracellular pH during “chemical hypoxia” in cultured rat hepatocytes. J. Clin. Invest. 83:386-396, 1989.
21. Grinwald, P. M. Calcium uptake during post ischemic reperfusion in the isolated rat heart: influence of extracellular sodium. J. Mol. Cell. Cardiol. 14:359-365, 1982.
22. Hearse, D. J. and Sutherland, F. J. The isolated blood and perfusion fluid perfused heart. Pharmacol. Res. 41(6):613-627, 2000.
23. Herman, B., Nieminen, A. L., Gores, G. J. and Lemasters, J. J. Irreversible injury in anoxic hepatocytes precipitated by an abrupt increase in plasma membrane permeability. FASEB J. 2:146-151, 1988.
24. Hoffstein, S., Gennaro, D. E., Fox, A. C., Hirsch, J., Streuli, F. and Weissman, G. Colloidal lanthanum as a marker for impaired plasma membrane permeability in ischemic dog myocardium. Am. J. Pathol. 79:207-218, 1975.
25. Howie, M. B., Dzwonczyk, R. and McSweeney, T. D. An evaluation of a new two-electrode myocardial electrical impedance monitor for detecting myocardial ischemia. Anesth. Analg. 92:12-18, 2001.
26. Ivorra, A., Gomez, R., Noguera, N., Villa, R., Sola, A., Palacios, L., Hotter, G. and Aguilo, J. Minimally invasive silicon probe for electrical impedance measurements in small animals. Biosensors Bioelectron. 391:391-399, 2003.
27. Jain, S. K., Schuessler, R. B. and Saffitz, J. E. Mechanisms of delayed electrical uncoupling induced by ischemic preconditioning. Circ. Res. 92:1138-1144, 2003.
28. Kleber, A. G., Riegger, C. B. and Janse, M. J. Electrical uncoupling and increase of extracellular resistance after induction of ischemia in isolated, arterially perfused rabbit papillary muscle. Circ. Res. 61:271-279, 1987.
29. Karthik Raghavan. A real-time approach in in-vivo phase measurements for the determination of volume in the murine heart. Thesis (M.S. in Engineering)—University of Texas at Austin, 2004.
30. Kottam, A. Determination of parasitic circuit elements in cardiac conductance catheters. Thesis (M.S. in Engineering)—University of Texas at Austin, 2003.
31. Kottam, A. and Pearce, J. A. Electric field penetration depth of myocardial surface catheters and the measurement of myocardial resistivity. Biomedical Sciences Instrumentation. 40:155-160, 2004.
32. Lee, H. C., Smith, N., Mohabir, R. and Clusin, W. T. Cytosolic calcium transients from the beating mammalian heart. Proc. Natl. Acad. Sci. USA. 84:7793-7797, 1987.
33. Lofgren, B. The electrical impedance of a complex tissue and its relation to changes in volume and fluid distribution; a study on rat kidneys. Acta. Physiol. Scand. Suppl. 81:1-51, 1951.
34. Lüss, H., Schäfers, M., Neumann, J., Hammel, D., Vahlhaus, C., Baba, H. A., Janssen, F., Scheld, H. H., Schober, O., Breithardt, G., Schmitz, W. and Wichter, T. Biochemical mechanisms of hibernation and stunning in the human heart. Cardiovasc. Res. 56:411-421, 2002.
35. Mangano, D. T. Perioperative cardiac morbidity. Anesthesiology. 72:153-184, 1990.
36. Marban, E., Koretsune, Y., Corretti, M., Chacko, V. P. and Kusuoka, H. Calcium and its role in myocardial cell injury during ischemia and reperfusion. Circulation. 80:IV17-IV22, 1989.
37. Nieminen, A. L., Gores, G. J., Wray, B. E., Tanaka, Y., Herman, B. and Lemasters, J. J. Calcium dependence of bleb formation and cell death in hepatocytes. Cell Calcium. 9:237-246, 1988.
38. Nishimura, Y. and Lemasters, J. J. Glycine blocks opening of a death channel in cultured hepatic sinusoidal endothelial cells during chemical hypoxia. Cell Death Differ. 8:850-858, 2001.
39. Olivetti, G., Lagrasta, C., Quaini, F., Ricci, R., Moccia, G., Capasso, J. M. and Anversa, P. Capillary growth in anemia-induced ventricular wall remodeling in the heart. Circ. Res. 65:1182-1192, 1989.
40. Owens, L. M., Fralix, T. A., Murphy, E., Cascio, W. E. and Gettes, L. S. Correlation of ischemia-induced extracellular and intracellular ion changes to cell-to-cell electrical uncoupling in isolated blood-perfused rabbit hearts. Circulation. 94:10-13, 1996.
41. Padilla, F., Garcia-Dorado, D., Rodriguez-Sinovas, A., Ruiz-Meana, M., Inserte, J. and Soler-Soler, J. Protection afforded by ischemic preconditioning is not mediated by effects on cell-to-cell electrical coupling during myocardial ischemia-reperfusion. Am. J. Physiol. Heart. Circ. Physiol. 285:H1909-H1916, 2003.
42. Panteghini, M. Acute coronary syndrome: biochemical strategies in the troponin era. Chest. 122:1428-1435, 2002.
43. Raby, K. E., Barry, J., Creager, M. A., Cook, E. F., Weisberg, M. C. and Goldman, L. Detection and significance of intraoperative and postoperative myocardial ischemia in peripheral vascular surgery. JAMA 268:222-227, 1992.
44. Raby, K. E., Brull, S. J., Timimi, F., Akhtar, S., Rosenbaum, S., Naimi, C. and Whittemore, A. D. The effect of heart rate control on myocardial ischemia among high-risk patients after vascular surgery. Anesth. Analg. 88:477-482, 1999.
45. Ramos, A., Raizer, A. and Marques, J. L. B. A new computational approach for electrical analysis of biological tissues. Bioelectrochemistry. 59:73-84, January 2003.
46. Rodriguez-Sinovas, A., Garcia-Dorado, D., Padilla, F., Inserte, J., Barrabes, J. A., Ruiz-Meana, M., Agullo, L. and Soler-Soler, J. Pre-treatment with the Na⁺/H⁺ exchange inhibitor cariporide delays cell-to-cell electrical uncoupling during myocardial ischemia. Cardiovasc. Res. 58:109-117, 2003.
47. Rodriguez-Sinovas, A., Garcia-Dorado, D., Pina, P., Ruiz-Meana, M. and Soler-Soler, J. Effect of sarcolemmal rupture on myocardial electrical impedance during oxygen deprivation. Am. J. Physiol. Heart. Circ. Physiol. 288:H1396-H1403, 2005.
48. Rodriguez-Sinovas, A., Garcia-Dorado, D., Ruiz-Meana, M. and Soler-Soler, J. Protective effect of gap junction uncouplers given during hypoxia against reoxygenation injury in isolated rat hearts. Am. J. Physiol. Heart. Circ. Physiol. 290:H648-H656, 2006.
49. Salazar, Y., et al., Transmural versus nontransmural in situ electrical impedance spectrum for healthy, ischemic, and healed myocardium. IEEE Trans. Biomed. Eng. 51:1421-1427, August 2004.
50. Schaefer, M., Gross, W., Ackemann, J. and Gebhard, M. M. The complex dielectric spectrum of heart tissue during ischemia. Bioelectrochemistry. 58:171-180, March 2002.
51. Schwan, H. P. Physical techniques in biological research. Electrophysiological Methods. vol. 6, pt. B, W. L. Nastuk, Ed., 1963, ch. 6.
52. Schwartzman, D., Chang, I., Michele, J. J., Mirotznik, M. S, and Foster, K. R. Electrical impedance properties of normal and chronically infarcted left ventricular myocardium. J. Interv. Card. Electrophysiol. 3:213-224, October 1999.
53. Steenbergen, C., Murphy, E., Levy, L. and London, R. E. Elevation in cytosolic free calcium concentration early in myocardial ischemia in perfused rat heart. Circ. Res. 60:700-707, 1987.
54. Steendijk, P., Mur, G., Van Der Velde, E. T. and Baan, J. The four electrode resistivity technique in anisotropic media: theoretical analysis and application of myocardial tissue in vivo. IEEE Trans. Biomed. Eng. 40:1138-1148, 1993.
55. Sugiura, H., Toyama, J., Tsuboi, N., Kamiya, K. and Kodama, I. ATP directly affects junctional conductance between paired ventricular myocytes isolated from guinea pig heart. Circ. Res. 66:1095-1102, 1990.
56. Surowiec, A., Stuchly, S. S, and Swarup, A. Postmortem changes of the dielectric properties of bovine brain tissues at low radiofrequencies. Bioelectromagnetics. 7:31-43, 1986.
57. Surowiec, A., Stuchly, S. S, and Swarup, A. Radiofrequency dielectric properties of animal tissues as a function of time following death. Phys. Med. Biol. 30:1131-1141, 1985.
58. Thom, T., Haase, N., Rosamond, W., Howard, V. J., Rumsfeld, J., Manolio, T., Zheng, Z., Flegal, K., O'Donnell, C., Kittner, S., Lloyd-Jones, D., Goff, D. and Hong, Y. Heart Disease and Stroke Statistics-2006 Update: A Report From the American Heart Association Statistics Committee and Stroke Statistics Subcommittee. Circulation. 113(6):E85-E151, 2006.
59. Urban, M. K., Gordon, M. A., Harris, S, N., O'Connor, T. and Barash, P. G. Intraoperative hemodynamic changes are not good indicators of myocardial ischemia. Anesth. Analg. 76:942-949, 1993.
60. van Oosterom, A., de Boer, R. W. and van Dam, R. T. Intramural resistivity of cardiac tissue. Med. Biol. Eng. Comput. 17:337-343, 1979.
61. Wtorek, J., et al., An averaging two electrode probe for monitoring changes in myocardial conductivity evoked by ischemia. IEEE Trans. Biomed. Eng. 49:240-246, March 2002.
62. Wu, J., McHowat, J., Saffitz, J. E., Yamada, K. A. and Con, P. B. Inhibition of gap junctional conductance by long-chain acylcarnitines and their preferential accumulation in junctional sarcolemma during hypoxia. Circ. Res. 72:879-889, 1993.
63. Zahrebelski, G., Nieminen, A. L., Al-Ghoul, K., Qian, T., Herman, B. and Lemasters, J. J. Progression of subcellular changes during chemical hypoxia to cultured rat hepatocytes: a laser scanning confocal microscopic study. Hepatology 21:1361-1372, 1995.

Although the invention has been described in detail in the foregoing embodiments for the purpose of illustration, it is to be understood that such detail is solely for that purpose and that variations can be made therein by those skilled in the art without departing from the spirit and scope of the invention except as it may be described by the following claims.

1. A method for identifying the viability of ischemic myocardium of a patient comprising the steps of:
measuring in real-time the ischemic myocardium of a beating or non-beating heart; and
determining in real-time whether the ischemic myocardium of the beating or non-beating heart is stunned or is nonviable.

2. A method as described in claim 1 wherein the measuring step includes the step of measuring the ischemic myocardium's electrical admittivity of the beating or non-beating heart.

3. An apparatus for identifying the viability of ischemic myocardium of a patient's heart comprising:
an electrode array having at least four electrodes for electrical communication with the heart which produces an array signal; and
a processor portion in communication with the array which receives the array signal and determines in real-time whether the ischemic myocardium of the heart is stunned or is nonviable.

4. An apparatus as described in claim 3 wherein the heart is nonviable if its relative permittivity is greater than about 19,000.

5. An apparatus for analyzing living tissue comprising:
an electrode array having at least four electrodes for electrical communication with the tissue which produces an array signal;
an admittance magnitude and phase detection circuit in communication with the array which receives the array signal from the array and produces a detection circuit signal corresponding to phase angle and magnitude of the array and signal; and
a processor in communication with the detection circuit which receives the detection circuit signal and determines permittivity and conductivity of the tissue in real-time.

6. An apparatus as described in claim 5 wherein the admittance magnitude detection circuit is an analog phase angle and magnitude detection circuit.

7. An apparatus as described in claim 6 wherein the admittance magnitude and phase angle detection circuit and the processor together are portable.

8. A method for analyzing living tissue comprising the steps of:
producing an array signal from an electrode array having at least four electrodes in electrical communication with the tissue;
receiving the array signal from the array at an admittance magnitude and phase angle detection circuit in communication with the array;
producing a detection circuit signal corresponding to phase angle and magnitude of the array signal with the phase angle and magnitude detection circuit; and
determining permittivity and conductivity of the tissue in real-time with a processor in communication with the detection circuit which receives the detection circuit signal.
